(19) United States
(12) Patent Application Publication
(10) Pub. No.: US 2007/0293575 A1
(43) Pub. Date: Dec. 20, 2007

(54) Compounds and methods for treatment of cancer-related anemia

(75) Inventors: Todd W. Seeley, Moraga, CA (US); David Y. Liu, Palo Alto, CA (US); Stephen J. Klaus, San Francisco, CA (US)

Correspondence Address: FIBROGEN, INC.;INTELLECTUAL PROPERTY DEPARTMENT, 225 GATEWAY BOULEVARD, SOUTH SAN FRANCISCO, CA, 94080, US

(73) Assignee: FibroGen, Inc., South San Francisco, CA (US)

(21) Appl. No.: 11/455,202

(22) Filed: Jun. 15, 2006

(51) Int. Cl. A61K 31/198

(52) U.S. Cl. 514/566

(57) ABSTRACT

The invention relates to methods and compounds for treating anemia of cancer. In particular, methods for treating anemia of cancer in a subject having cancer, and methods for increasing reticulocytes, increasing hemoglobin, increasing hematocrit, and increasing red blood cell count in subjects having anemia of cancer, wherein such subjects are refractory to treatment with recombinant human erythropoietin (EPO) are encompassed herein.

### FIELD OF THE INVENTION

The invention relates to methods and compounds for treating anemia of cancer. In particular, methods for treating anemia of cancer in a subject having cancer, and methods for increasing reticulocytes, increasing hemoglobin, increasing hematocrit, and increasing red blood cell count in subjects having anemia of cancer, wherein such subjects are refractory to treatment with recombinant human erythropoietin (EPO) are encompassed herein.

### BACKGROUND OF THE INVENTION

A frequent feature in cancer is anemia. In the cancer patient, anemia is the most common cancer-associated morbidity, and is also an adverse prognostic factor for survival independent of tumor type. (Spivak (2005) Nat Rev Cancer 5:543-555.) The mechanisms underlying cancer-associated anemia are diverse and can occur as a direct effect of the cancer or due to various factors produced by or in response to the cancer. The hematological features in patients with cancer-related anemia depend to some extend on the underlying type of malignant disease. However, for any degree of anemia, the serum erythropoietin (EPO) concentration is lower in cancer patients than in patients with, e.g., iron-deficiency anemia; and the expected inverse linear relation between serum EPO and hemoglobin is absent in cancer patients. (Miller et al. (1990) N Eng J Med 322(24):1689-1692.)

No adequate therapy exists for treating anemia of cancer. Attempts to treat anemia of cancer by administering recombinant human erythropoietin (rHuEPO) have produced limited benefit. Although rHuEPO can ameliorate anemia and reduce transfusion requirement in some cancer patients, the response rate in published studies range from a low of 32% to a high of 85%. (Spivak (1994) Blood 84:997-1004.) Thus, rHuEPO does not provide improved quality of life for a substantial number of patients having anemia. In particular, there is a need for methods for treating anemia of cancer in patients who are refractory to treatment with recombinant human erythropoietin (EPO).

### SUMMARY OF THE INVENTION

The present invention provides methods for treating or preventing anemia of cancer of cancer-related anemia in a subject in need. In various embodiments, the methods comprise treating or preventing anemia of cancer in a subject, the method comprising administering to the patient an effective amount of an agent that inhibits HIF hydroxylase activity.

For purposes of this invention, a subject in need is a cancer patient who has or who is at risk for having anemia of cancer of cancer-related anemia.

A subject suitable for treatment with the present methods and compounds is a subject who is refractory to or is at risk for being refractory to recombinant human EPO therapy. Whether or not a subject is refractory to recombinant human EPO therapy can be determined by an assessment of the subject's response or predicted response to treatment with recombinant human EPO. For example, in particular embodiments, the subject is a subject refractory to treatment with rhEPO if the subject displays an increase in hemoglobin concentration of less than 2 g/dl, or fails to reach levels of at least 12 g/dl, after undergoing a regimen of dosing with rhEPO. In a particular embodiment, the response desired upon treatment with recombinant EPO can be defined as an increase in hemoglobin of at least 2 g/dl over a twelve (12) week dosing regimen. If a subject does not display such a response within the required period of time, that subject is deemed refractory to treatment with recombinant human EPO. (See, e.g., Ludwig et al. (1994) Blood 84:1056-1063.)

The value of the desired increase in hemoglobin, which can be used to determine whether or not a particular subject is refractory to recombinant human EPO treatment, may vary depending on a number of factors, including age and gender. Thus, in various embodiments of the present invention, a subject can be a subject refractory to treatment with recombinant human EPO if treatment with recombinant human EPO according to a specific dosing regimen fails to increase the subject's hemoglobin level by at least 0.1-5.0 g/dL. In some embodiments, a subject is refractory to treatment with recombinant human EPO if such treatment fails to increase the subject's hemoglobin level by an amount of at least 0.2-5.0, 0.5-5.0, 1.0-5.0, 1.5-5.0, 2.0-5.0, 3.0-5.0 or 4.0-5.0 g/dL. According to further embodiments, the subject is a subject refractory to therapy using recombinant human EPO if such therapy fails to increase the subject's hemoglobin level by an amount of at least 0.2-2.5, 0.4-2.5, 0.6-2.5, 0.8-2.5, 1.0-2.5, 1.2-2.5, 1.4-2.5, 1.6-2.5, 1.8-2.5, or 2-2.5 g/dL, respectively. Finally, in certain embodiments, the subject is a subject refractory to recombinant human EPO therapy if such therapy fails to raise the subject's hemoglobin to at least desired levels of at least 1.0-2.0, 1.1-2.0, 1.2-2.0, 1.3-2.0, 1.4-2.0, 1.5-2.0, 1.6-2.0, 1.6-2.0, 1.7-2.0, 1.8-2.0, or 1.9-2.0 g/dL, respectively.

Other parameters can be used to determine whether a particular subject is refractory to recombinant human EPO therapy. For example, current guidelines relating to rhEPO administration define target hemoglobin levels for an adult subject as 12 gm/dL. Therefore, in one embodiment, a subject is a subject refractory to recombinant human EPO therapy if treatment with acceptable doses over a specific period of time fail to increase hemoglobin to at least 12 gm/dL. In other embodiments, a subject is a subject refractory to recombinant human EPO therapy if treatment with acceptable doses over a specific period of time fail to increase hemoglobin to at least 10 gm/dL, or at least 11 gm/dL.

Similarly, current guidelines for recombinant human EPO administration define a target hematocrit level for an adult subject as a hematocrit of 35%. Thus, it is contemplated that, in certain embodiments, a subject is refractory to treatment with recombinant human EPO if dosing with recombinant human EPO fails, over a specified period of time, to raise the subject's hematocrit level to at least 36%. In various embodiments, the subject is refractory to treatment with recombinant human EPO if a recombinant human EPO dosing regimen fails to raise the subject's hematocrit to at least 30%, at least 36%, and at least 42%. respectively.

It is understand that, in view of the discussion, supra, subjects suitable for treatment with the present methods and compounds, e.g., subjects refractory to treatment with recombinant human EPO, methods and compounds provided herein for treating such subjects specifically encompass methods and compounds capable of increasing the subject's hematocrit, hemoglobin, red blood cell count, reticulocyte count, etc., to desired or recommended levels. Failure to meet desired levels of any of these factors through dosing with recombinant human EPO can also be used singly or in combination, to determine whether a subject is or may be refractory to treatment with recombinant human EPO. For example, in one aspect, subject is a refractory subject suitable for treatment using the present methods if, after two weeks of therapy, the subject has a serum EPO level of or less than 100 mU/mL, and has demonstrated an increase in hemoglobin of less than 0.5 g/dL. In another aspect, the subject is a subject refractory to recombinant human EPO therapy if, after two weeks of recombinant human EPO therapy, the subject displays a serum ferritin level of greater than or equal to 400 ng/ml.

The invention provides in one embodiment a method for increasing hemoglobin in a cancer patient having or at risk for having anemia of cancer, the method comprising administering to the subject an effective amount of an agent that inhibits HIF hydroxylase activity, wherein the patient is refractory to or is at risk for being refractory to treatment with recombinant human EPO. In various embodiments, the methods comprise increasing hemoglobin by at least 0.2-2.5, 0.4-2.5, 0.6-2.5, 0.8-2.5, 1.0-2.5, 1.2-2.5, 1.4-2.5, 1.6-2.5, 1.8-2.5, or 2-2.5 g/dL, g/dL, respectively; or to levels of at least 10, 11, 12, 13, or 14 g/dL, respectively.

The invention further provides methods for increasing hematocrit in a subject having anemia of cancer, the method comprising administering to the subject an effective amount of an agent that inhibits HIF hydroxylase activity, wherein the subject is refractory to or is at risk for being refractory to EPO treatment. Embodiments in which the increase in hematocrit achieved using the present methods and compounds is an increase in hematocrit to at least 30, 33, 36, 39, and 42%, respectively.

Methods for increasing the RBC count in a subject having anemia of cancer, the methods comprising administering to the subject an effective amount of an agent that inhibits HIF hydroxylase activity, wherein the subject is refractory to or is at risk for being refractory to EPO treatment are specifically encompassed by the present invention. A normal red blood cell count for adult males is 4.2-5.4 million red blood cells per milliliter; for adult females if 3.6-5.0 million red blood cells per milliliter; and for children is 4.6-4.8 million red blood cells per milliliter. Particular embodiments in which the RBC count is increased to levels at or near normal levels are specifically encompassed herein.

The present invention also contemplates methods for increasing reticulocytes in a subject having anemia of cancer, the methods comprising administering to the subject an effective amount of an agent that inhibits HIF hydroxylase activity, wherein the subject is refractory to or is at risk for being refractory to EPO treatment. Further embodiments in which reticulocytes are increased to levels of at least 0.5%, 1%, 1.5%, 2%, or 2.5% of circulating erythrocytes, are encompassed herein.

It should be noted that, while medical applications with humans are clearly foreseen, veterinary applications are also encompassed herein. In particular, a distinct advantage to the present compounds is that they can be administered to any animal subject without risk of an adverse immune response. Due to the cost, in time and effort, associated with production of recombinant proteins, non-human recombinant erythropoeitins are not commonly available for therapeutic use. Therefore, there is a need in the art for available therapies for treatment of anemia of cancer in non-human subjects. The present invention answers this need by providing methods for treating or preventing anemia of cancer in non-human subjects, preferably, non-human mammalian subjects, and, most preferably, cats and dogs, the methods comprising administering to the non-human subject having cancer an effective amount of an agent that inhibits HIF hydroxylase activity.

In various embodiments, an agent for use in the present methods is a 2-oxoglutarate mimetic. In certain embodiments, the agent used in the present methods is a compound selected from the group consisting of the compounds of Formula I, Formula II, Formula III, and Formula IV. Formula I includes, but is not limited to, compounds of Formulae Ia, Ib, Id, and Ie; compounds of Formula Ie include, but are not limited to, compounds of Formula Ie(i), Ie(ii), Ie(iii), and Ie(iv). Formula III includes but is not limited to, the compounds of Formula IIIa.

In particular embodiments, an agent of the present invention is selected from the group consisting of a pyridine-2-carboxamide, a quinoline-2-carboxamide, an isoquinoline3-carboxamide, a cinnoline-3-carboxamide, a beta-carboline-3-carboxamide, and a 4-oxo-[1,10]-phenanthroline.

In particular embodiments, an agent for use in the present methods is selected from group consisting: Compound A (1-Chloro-4-hydroxy-isoquinoline-3-carbonyl)-amino]-acetic acid; Compound B (S)-2-[(4-Hydroxy-7-phenoxy-isoquinoline-3-carbonyl)-amino]-propionic acid; Compound C {[4-Hydroxy-7-(4-methoxy-phenoxy)-isoquinoline-3-carbonyl]-amino}-acetic acid; Compound D [(4-Hydroxy-1-methyl-7- phenoxy-isoquinoline-3-carbonyl)-amino]-acetic acid; and Compound E [7-(4-Fluoro-phenoxy)-4-hydroxy-isoquinoline-3-carbonyl]-amino-acetic acid.

Pharmaceutical compositions or medicaments effective for treating or preventing anemia of cancer in a subject having cancer, wherein the subject is refractory or is at risk for being refractory to recombinant human EPO therapy, are provided herein. In various embodiments, the compositions comprise an effective amount of an agent that inhibits HIF hydroxylase activity an a carrier.

In various embodiments of the present methods, the agent is administered orally, systemically, by injection, and intravenously.

### BRIEF DESCRIPTION OF THE DRAWINGS

FIG. 1 sets forth data showing methods and compounds of the present invention increased hematocrit in an animal model of cancer of anemia.

FIG. 2 sets for data showing methods and compounds of the present invention increased hematocrit in an animal model of anemia of cancer.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a, ” “an,” and “the ” include plural references unless context clearly dictates otherwise. Thus, for example, a reference to “a fragment” includes a plurality of such fragments; a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Hardman, J. G., Limbird, L. E., and Gilman, A. G., eds. (2001) The Pharmacological Basis of Therapeutics, 10^{th} ed., McGraw-Hill Co.; Colowick, S. et al., eds., Methods in Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2^{nd} ed., Springer Verlag.

### Invention

The present invention relates to the discovery that administration of an agent that inhibits HIF hydroxylase activity is therapeutically effective in treating cancer-related anemia in subjects having cancer, wherein such subjects are refractory to or are at risk for being refractory to treatment with recombinant human EPO.

The present methods and compounds can be used to treat or prevent cancer-related anemia in cancer patients having or at risk for having anemia of cancer.

A subject suitable for treatment with the present methods and compounds is a subject who is refractory to or is at risk for being refractory to recombinant human EPO therapy. Whether or not a subject is refractory to recombinant human EPO therapy can be determined by an assessment of the subject's response or predicted response to treatment with recombinant human EPO. For example, in one particular embodiment, the response desired upon treatment with recombinant EPO can be defined as an increase in hemoglobin of at least 2 g/dl over a twelve (12) week dosing regimen. If a subject does not display such a response within the required period of time, that subject is deemed refractory to treatment with recombinant human EPO. (See, e.g., Ludwig et al. (1994) Blood 84:1056-1063)

The value of the desired increase in hemoglobin, used to determine whether or not a particular subject is refractory to EPO treatment, may vary depending on a number of factors, including age and gender. Various factors, including hemoglobin or Hb concentration, serum EPO levels, and hematocrit, can be used singly or in combination to assess whether a particular subject is refractory to or at risk for being refractory to recombinant human EPO therapy.

Thus, in various embodiments of the present invention, a subject can be a subject refractory to treatment with recombinant human EPO if treatment with recombinant human EPO according to a specific dosing regimen fails to increase the subject's hemoglobin level by at least 0.1-5.0 g/dL. In some embodiments, a subject is refractory to treatment with recombinant human EPO if such treatment fails to increase the subject's hemoglobin level by an amount of at least 0.2-5.0, 0.5-5.0, 1.0-5.0, 1.5-5.0, 2.0-5.0, 3.0-5.0 or 4.0-5.0 g/dL. According to further embodiments, the subject is a subject refractory to therapy using recombinant human EPO therapy if such therapy fails to increase the subject's hemoglobin level by an amount of at least 0.2-2.5, 0.4-2.5, 0.6-2.5, 0.8-2.5, 1.0-2.5, 1.2-2.5, 1.4-2.5, 1.6-2.5, 1.8-2.5, or 2-2.5 g/dL, respectively. Finally, in certain embodiments, the subject is a subject refractory to recombinant human EPO therapy if such therapy fails to raise the subject's hemoglobin to at least desired levels of at least 1.0-2.0, 1.1-2.0, 1.2-2.0, 1.3-2.0, 1.4-2.0, 1.5-2.0, 1.5-2.0, 1.7-2.0, 1.8-2.0, or 1.9-2.0 g/dL, respectively.

Other parameters can be used to determine whether a particular subject is refractory to recombinant human EPO therapy. For example, current guidelines relating to rhEPO administration define target hemoglobin levels for an adult subject as 12 gm/dL. Therefore, in one embodiment, a subject is a subject refractory to recombinant human EPO therapy if treatment with acceptable doses over a specific period of time fail to increase hemoglobin to at least 12 gm/dL. In other embodiments, a subject is a subject refractory to recombinant human EPO therapy if treatment with acceptable doses over a specific period of time fail to increase hemoglobin to at least 10 gm/dL., or at least 11 gm/dl.

Similarly, current guidelines for recombinant human EPO administration define a target hematocrit level for an adult subject as a hematocrit of 36%. Thus, it is contemplated that, in certain embodiments, a subject is refractory to treatment with recombinant human EPO if dosing with recombinant human EPO fails, over a specified period of time, to raise the subject's hematocrit level to at least 36%. In various embodiments, the subject is refractory to treatment with recombinant human EPO if a recombinant human EPO dosing regimen fails to raise the subject's hematocrit to at least 30%, at least 33%, at least 36%, at least 39%, and at least 42%, respectively.

It is noted that various factors, including hemoglobin or Hb concentration, hematocrit, reticulocyte or RBC count, serum ferritin levels, and serum EPO levels can be measured by any of the methods available in the art and can be used singly or in combination to determine whether a particular subject is refractory, or may be refractory, to treatment with recombinant human EPO. For example, in one embodiment, it is contemplated that serum EPO levels and Hb concentration are used in combination to identify subjects suitable for treatment with the present methods and compounds. In a specific embodiment, the subject is a refractory subject suitable for treatment using the present methods if, after two weeks of therapy, the subject has demonstrated an increase in hemoglobin of less than 0.5 g/dL. In another aspect, the subject is a subject refractory to recombinant human EPO therapy if, after two weeks of recombinant EPO therapy, the subject has a high serum ferritin level, for example, a serum ferritin level of greater than or equal to 400 ng/ml.

In particular, it is demonstrated herein that HIF hydroxylase inhibitors effectively treated anemia of cancer in established xenograft models of human cancer. Xenograft models of human cancer have played a significant role in development of cancer-related therapies and constitute a predictive indicator of clinical activity.

The present invention demonstrates that methods and compounds of the present invention increased EPO levels, red blood cell counts, hemoglobin levels, and hematocrit in various animal models of anemia of cancer. The present invention further demonstrates that methods and compounds of the present invention were effective at treating and preventing the development of anemia of cancer or cancer-related anemia. The invention provides in one embodiment a method for increasing hemoglobin in a cancer patient having or at risk for having anemia of cancer, the method comprising administering to the subject an effective amount of an agent that inhibits HIF hydroxylase activity, wherein the patient is refractory or is at risk for being refractory to treatment with recombinant human EPO. In various embodiments, the methods comprise increasing hemoglobin by at least 0.2-2.5, 0.4-2.5, 0.6-2.5, 0.8-2.5, 1.0-2.5, 1.2-2.5, 1.4-2.5, 1.6-2.5, 1.8-2.5, or 2-2.5 g/dL, g/dL, respectively; or to levels of at least 10, 11, 12, 13or 14 g/dL, respectively.

The invention further provides methods for increasing hematocrit in a subject having anemia of cancer, the method comprising administering to the subject an effective amount of an agent that inhibits HIF hydroxylase activity, wherein the subject is refractory or is at risk for being refractory to EPO treatment. Embodiments in which the increase in hematocrit achieved using the present methods and compounds is an increase in hematocrit to at least 30, 33, 36, 39, and 42%, respectively.

Methods for increasing the RBC count in a subject having anemia of cancer, the methods comprising administering to the subject an effective amount of an agent that inhibits HIF hydroxylase activity, wherein the subject is refractory or is at risk for being refractory to EPO treatment, are specifically encompassed by the present invention. Particular embodiments in which the RBC count is increased to levels at or near normal levels are specifically encompassed herein. A normal red blood cell count for adult males 4.2-5.4 million red blood cells per milliliter; for adult females 3.6-5.0 million red blood cells per milliliter; and for children is 4.6-4.8 million red blood cells per milliliter.

The present invention also contemplates methods for increasing reticulocytes in a subject having anemia of cancer, the methods comprising administering to the subject an effective amount of an agent that inhibits HIF hydroxylase activity, wherein the subject is refractory or is at risk for being refractory to EPO treatment. Further embodiments in which reticulocytes are increased to levels of at least increased to levels of at least 0.5%, 1%, 1.5%, 2%, or 2.5% of circulating erythrocytes, are encompassed herein.

### Cancers

The present invention provides methods for treating or preventing anemia of cancer in a subject in need of such treatment. In various embodiments, the subject in need is a human subject. In preferred embodiments, the subject is a human subject having cancer, wherein the subject is refractory to or is at risk to being refractory to recombinant human EPO therapy.

It is contemplated herein that the subject is a subject having or at risk for having a cancer selected from the non-limiting examples of cancers provided infra, and has developed or is at risk for developing cancer-related anemia in conjunction therewith.

The cancer can be a cancer selected from the group consisting of lung cancer, including non-small cell lung cancer and large cell carcinoma types, as well as small cell lung cancer; colon cancer, including colon metastasized to liver and including colorectal cancers; breast cancer; ovarian cancer; kidney or renal cancers, including, for example, renal cell carcinomas; cancer of the bladder; liver cancer, including, for example, hepatocellular carcinomas; cancer of the gastrointestinal tract, including rectal, esophageal, pancreatic, and stomach cancer; gynecological cancers, including cervical, uterine, and endometrial cancers; prostate cancer or testicular cancer; nasopharyngeal cancer; thyroid cancer, for example, thyroid papillary carcinoma; cancer of the head, mouth, lips, eye, neck, or brain; nervous system, including neuroblastomas; skin, including melanomas, and sarcomas (including, for example, osteosarcomas and Ewing's sarcomas). Carcinomas include, but are not limited to, adenocarcinomas and epithelial carcinomas.

Hematological malignancies are cancers that affect blood, bone marrow, and lymph nodes and include leukemia, lymphomas, and myeloma. Such malignancies are typically associated with formation of non-solid tumors or non-solid tumor masses. Underlying genetic alterations, particularly chromosomal translocations, are a common cause of hematological malignancy, affecting the approach to diagnosis and treatment of these disorders.

Leukemia is characterized by an abnormal proliferation of white blood cells (leukocytes) or myeloid precursors. Displacement of normal marrow with increasing numbers of malignant cells results in a lack of blood platelets (thrombocytopenia), which are important in blood clotting, and red blood cells, which provide oxygen to the tissues of the body. Thus, patients with leukemia may bruise easily, bleed excessively, and suffer from anemia. Additionally, the number of functional white blood cells is often reduced, making leukemia patients susceptible to infection. Types of leukemia include acute lymphoblastic leukemia (ALL), characterized by overproduction of malignant and immature white blood cells, chronic lymphocytic leukemia (CLL); acute and chronic myelogenous leukemia (AML and CML, respectively), characterized by increased myeloid precursors in the blood and bone marrow; hairy cell leukemia, a rare leukemia also known as leukemic reticuloendotheliosis; and myelogenous leukemia. Leukemias may originate from myeloid bone marrow or lymph nodes. Leukemias may be acute, exhibited by maturation arrest at a primitive state of development, and chronic, exhibited by excess accrual of mature lymphoid or myeloid cells.

Lymphomas originate in cells, primarily lymphocytes, of the reticuloendothelial system, which includes the lymph nodes and lymphatic organs such as spleen, thymus, tonsils, etc. Lymphomas include Hodgkin's lymphoma, characterized by the presence of large, often binucleated malignant cells known as Reed-Sternberg cells; and non-Hodgkin lymphoma, which includes a variety of lymphomas in which Reed-Sternberg cells are absent.

Multiple myeloma (MM) is a cancer of post-germinal center B-lymphocytes, and can affect several organs due to proliferation of the cancer cells, deposition of antibody, and overproduction of cytokines. Common ailments associated with MM include renal failure, polyneuropathy, bone lesions, and anemia. The anemia is usually normocytic and normochronic, and results from replacement of normal bone marrow by infiltrating tumor cells and inhibition of normal red blood cell production by cytokines.

Hematological malignancies include, but are not limited to, leukemias, including, but not limited to, acute myeloid leukemia (AML), chronic myelogenous leukemia (CML), acute lymphoblastic or precursor lymphoblastic leukemia, chronic lymphocytic leukemia (CLL), and hairy cell leukemia; lymphomas, e.g., mature B cell neoplasms, mature T cell and natural killer (NK) cell neoplasms, Hodgkin's lymphoma, immunodeficiency-associated lymphoproliferative disorders, and histiocytic and dentritic cell neoplasms, etc.; and myelomas, such as multiple myelomas.

### Methods

Various methods are provided herein. In one aspect, the methods comprise administering to a subject an agent that inhibits HIF hydroxylase activity. HIF hydroxylase activity can include, e.g., the activity of any enzyme selected from the group consisting of HIF prolyl hydroxylase, HIF asparaginyl hydroxylase, and HIF lysyl hydroxylase. In preferred embodiments, the enzyme is a HIF prolyl hydroxylase enzyme, e.g., EGLN-1, EGLN-2, EGLN-3,etc. (See, e.g., Taylor (2001) Gene 275:125-132; Epstein et al. (2001) Cell 107:43-54; and Bruick and McKnight (2001) Science 294:1337-1340.)

A HIF hydroxylase is any enzyme capable of hydroyxlating a residue in the HIF protein. HIF hydroxylases include HIF prolyl hydroxylases. In certain embodiments, the residue hydroxylated by HIF prolyl hydroxylase includes the proline within the motif LXXLAP, e.g., as occurs in the human HIF-1α native sequence at L₃₉₇TLLAP and L₅₅₉EMLAP. HIF prolyl hydroxylase includes members of the Egl-Nine (EGLN) gene family described by Taylor (2001), Gene 275:125-132), and characterized by Aravind and Koonin (2001), Genome Biol 2:RESEARCH0007), Epstein et al. (2001, Cell 107:43-54), and Bruick and McKnight (2001), Science 294:1337-1340). Examples of HIF prolyl hydroxylase enzymes include human SM-20 (EGLN1) (GenBank Accession No. AAG33965; Dupuy et al. (2000) Genomics 69:348-54), EGLN2 isoform 1 (GenBank Accession No. CAC42510; Taylor, supra), EGLN2 isoform 2 (GenBank Accession No. NP_{—}060025), and EGLN3 (GenBank Accession No. CAC42511; Taylor, supra); mouse EGLN1 (GenBank Accession No. CAC42515), EGLN2 (GenBank Accession No. CAC42511), and EGLN3 (SM-20) (GenBank Accession No. CAC42517); and rat SM-20 (GenBank Accession No. AAA19321). Additionally, HIF prolyl hydroxylase may include Caenorhabditis elegans EGL-9 (GenBank Accession No. AAD56365) and Drosophila melanogaster CG1114 gene product (GenBank Accession No. AAF52050). HIF prolyl hydroxylase also includes any fragment of the foregoing full-length proteins that retain at least one structural or functional characteristic.

An agent that inhibits HIF hydroxylase activity is any agent that reduces or otherwise modulates the activity of a HIF hydroxylase enzyme. In particular embodiments of the present invention, the agent that inhibits HIF hydroxylase activity is a structural mimetics of 2-oxoglutarate. Such compounds may inhibit the target 2-oxoglutarate dioxygenase enzyme family member competitively with respect to 2-oxoglutarate. (Majamaa et al. (1984) Eur J Biochem 138:239-245; and Majamaa et al. (1985) Biochem J 229:127-133.) Hydroxylase inhibitors specifically contemplated for use in the present methods are described, e.g., in Majamaa et al., supra; Kivirikko and Myllyharju (1998) Matrix Biol 16:357-368; Bickel et al. (1998) Hepatology 28:404-411; Friedman et al. (2000) Proc Natl Acad Sci USA 97:4736-4741; Franklin (1991) Biochem Soc Trans 19):812 815; Friedman et al. (2000) Biochem J 353:333-338; and International Publication Nos. WO 03/053977 and WO 03/049686, each incorporated by reference herein in its entirety. Exemplary HIF prolyl hydroxylase inhibitors, including Compound A (1-Chloro-4-hydroxy-isoquinoline-3-carbonyl)-amino]-acetic acid; Compound B (S)-2-[(4-Hydroxy-7phenoxy-isoquinoline-3-carbonyl)-amino]-propionic acid; Compound C {[4-Hydroxy-7-(4-methoxy-phenoxy)-isoquinoline-3-carbonyl]-amino}-acetic acid; Compound D [(4-Hydroxy-1-methyl-7-phenoxy-isoquinoline-3-carbonyl)-amino]-acetic acid; and Compound E [7-(4-Fluoro-phenoxy)-4-hydroxy-isoquinoline-3-carbonyl]-amino-acetic acid are used in the present examples to demonstrate the methods of the invention described herein.

### Compounds

In preferred methods, the present methods comprise administering to a subject an effective amount of a compound that stabilizes HIFα. Exemplary compounds are disclosed in, e.g., International Publication No. WO 03/049686, International Publication No. WO 03/053997, International Publication No. WO 04/108121, and International Publication WO 04/108681, each of which is incorporated herein by reference in their entireties.

For example, International Publication No. WO 03/049686, International Publication No. WO 03/053997, International Publication No. WO 04/108121, and International Publication No. WO 04/108681 disclose exemplary compounds according to Formula I, below. These compounds include, but are not limited to, compounds of Formulae Ia, Ib, Ic, and Id. Further exemplary compounds are according to Formula Ie, including, but not limited to, compounds of Formulae Ic(i), Ie(i), Ie(iii), and Ie(iv), as described below. International Publication No. WO 03/049686 and International Publication No. WO 03/053997 disclose exemplary compounds according to Formula II, below. Exemplary compounds according to Formula III, shown below, are disclosed in International Publication No. WO 03/049686, International Publication No. WO 03/053997, and International Publication No. WO 04/108121. These compounds include, but are not limited to, compounds of Formula IIIa. Further exemplary compounds are according to Formula IV, as described below.

In certain embodiments, a compound of the invention is a compound that inhibits HIF hydroxylase activity. In various embodiments, the activity is due to a HIF prolyl hydroxylase, such as, for example, EGLN1, EGLN2, or EGLN3, etc. In other embodiments, the activity is due to a HIF asparaginyl hydroxylase, such as, for example, including, but not limited to, FIH. A preferred compound of the invention is a compound that inhibits HIF prolyl hydroxylase activity. The inhibition can be direct or indirect, can be competitive or non-competitive, etc.

In one aspect, a compound of the invention is any compound that inhibits or otherwise modulates the activity of a 2-oxoglutarate dioxygenase enzyme. 2-oxoglutarate dioxygenase enzymes include, but are not limited to, hydroxylase enzymes. Hydroxylase enzymes hydroxylate target substrate residues and include, for example, prolyl, lysyl, asparaginyl (aspartyl) hydroxylases, etc. Hydroxylases are sometimes described by target substrate, e.g., HIF hydroxylases, procollagen hydroxylases, etc., and/or by targeted residues within the substrate, e.g., prolyl hydroxylases, lysyl hydroxylases, etc., or by both, e.g., HIF prolyl hydroxylases, procollagen prolyl hydroxylases, etc. Representative 2-oxoglutarate dioxygenase enzymes include, but are not limited to, HIF hydroxylases, including HIF prolyl hydroxylases, e.g., EGLN1, EGLN2, and EGLN3, HIF asparaginyl hydroxylases, e.g., factor inhibiting HIF (FIH), etc.; procollagen hydroxylases, e.g., procollagen lysyl hydroxylases, procollagen prolyl hydroxylases, e.g., procollagen prolyl 3-hydroxylase, procollagen prolyl 4-hydroxylase α(I) and α(II), etc.; thymine 7-hydroxylase; aspartyl (asparaginyl) β-hydroxylase; ε-N-trimethyllysine hydroxylase; γ-butyrobetaine hydroxylase, etc. Although enzymatic activity can include any activity associated with any 2-oxoglutarate dioxygenase, the hydroxylation of amino acid residues within a substrate is specifically contemplated. Although hydroxylation of proline and/or asparagine residues within a substrate is specifically included, hydroxylation of other amino acids is also contemplated.

In one aspect, a compound of the invention that shows inhibitory activity toward one or more 2-oxoglutarate dioxygenase enzyme may also show inhibitory activity toward one or more additional 2-oxoglutarate dioxygenase enzymes, e.g., a compound that inhibits the activity of a HIF hydroxylase may additionally inhibit the activity of a collagen prolyl hydroxylase, a compound that inhibits the activity of a HIF prolyl hydroxylase may additionally inhibit the activity of a HIF asparaginyl hydroxylase, etc.

In some aspects, compounds of the present invention include, for example, structural mimetics of 2-oxoglutarate. Such compounds may inhibit the target 2-oxoglutarate dioxygenase enzyme family member competitively with respect to 2-oxoglutarate. (Majamaa et al. (1984) Eur J Biochem 138:239-245; and Majamaa et al. Biochem J 229:127-133.)

In certain embodiments, a compound of the present invention is a compound of Formula I. In particular embodiments, the 2-oxoglutarate mimetic is a pyridine-2-carboxamide including, but not limited to, compounds of Formula I. In particular embodiments, the 2oxoglutarate mimetic is a quinoline-2-carboxamide including, but not limited to, compounds of Formula Ia. In other embodiments, the 2-oxoglutarate mimetic is an isoquinoline-3-carboxamide including, but not limited to, compounds of Formula Ib. In additional embodiments, the 2- oxoglutarate mimetic is a cinnoline-3-carboxamide including, but not limited to, compounds of Formula Ic, or is a beta-carboline-3-carboxamide including, but not limited to, compounds of Formula Id.

As stated above, in certain embodiments, a compounds of the present invention is a compound of Formula I

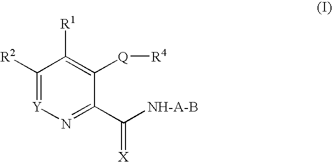

wherein
A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C_{f}H_{(2f+1-g)}Hal_{g}, (C₁-C₆)-fluoroalkoxy, (C₁-C₈)-fluoroalkenyloxy, (C₁-C₈)-fluroalkynyloxy, —OCF₂Cl, —O—CF_{X}—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N-(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N-(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)}Hal_{g}, —OCF₂Cl, —O—CF₂—CHFCl, (C₁C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N-(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N-(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —OH₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO2, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂—G carboxyl radical, where G is a radical of an alcohol G—OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds/ (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁C₁₂)-alkoxy-(C₁C₁₂)-alkoxy, (C₆-C₁₂)-aryloxy, (C₂-C₁₆)-aralkyloxy, (C₁-C₈)-hydroxyalkyl, —O—[CH₂]ₓ—C_{f}H_{(2f+1-g)}—F_{g}, —OCR₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆) aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di(C₁-C₁₂)-alkylcarbamoyl, N-(C₃-C₈)-cycloalkyl-carbamoyl, N-(C₆-C₁₆)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, N-((C₁-C₁₀)-alkoxy-(C₁-C₁₀)-alkyl)-carbamoyl, N-((C₆-C₁₂)-aryloxy-(C₁-C₁₀)alkyl)-carbamoyl, N-((C₇-C₁₆)-aralkyloxy-(C₁-C₁₀)-alkyl)-carbamoyl, N-(C₁-C₁₀)-alkyl-N-((C₁-C₁₀)-alkoxy-(C₁-C₁₀)-alkyl)-carbamoyl, N-(C₁-C₁₀)-alkyl-N-((C₆-C₁₆)-aryloxy-(C₁-C₁₀)-alkyl)-carbamoyl, N-(C₁-C₁₀)-alkyl-N-((C₇-C₁₆)-aralkyloxy-(C₁-C₁₀)-alkyl)-carbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C-C₁₆)-aralkylcarbamoyloxy, N-(C₁-C₁₀)-alkyl-N-(C₆-C₁₂)-arylcarbamoyloxy, N(C₁-C₁₀)-alkyl-N-(C₇-C₁₆)-aralkylcarbamoyloxy, N-((C₁-C₁₀)-alkyl)-carbamoyloxy, N-((C₆-C₁₂)-aryloxy-(C₁-C₁₀)-alkyl)-carbamoyloxy, N-((C₇-C₁₆)-aralkyloxy-(C₁-C₁₀)-alkyl)-carbamoyloxy, N-(C₁-C₁₀)-alkyl-N-((C₁-C₁₀)-alkoxy-(C₁-C₁₀)-alkyl)-carbamoyloxy, N-(C₁-C₁₀)-alkyl-N-((C₆-C₁₂)-aryloxy-(C₁-C₁₀)-alkyl)-carbamoyloxy, N-(C₁-C₁₀)-alkyl-N-((C₁-C₁₆)-aralkyloxy-(C₁-C₁₀)-alkyl)-carbamoyloxy, amino, (C₁-C₁₂)-alkylamino, di(C₁-C₁₂)-alkylamino, (C₃-C₁₂)-cycloalkylamino, (C₂-C₁₂)-alkenylamino, (C₂-C₁₂)-alkynylamino, N-(C₆-C₁₂)-arylamino, N-(C-C₁₁)-aralkylamino, N-alkyl-aralkylamino, N-alkyl-arylamino, (C₁-C₁₂)-alkoxyamino, (C_{1-C}₁₂)-alkoxy-N-(C₁-C₁₀)-alkylamino, (C₁-C₁₂)-alkylcarbonylamino, (C₃-C₈)-cycloalkylcarbonylamino, (C₆-C₁₂) arylcarbonylamino, (C₇-C₁₆)-aralkylcarbonylamino, (C₁-C₁₂)-alkylcarbonyl-N-(C₁-C₁₀)-alkylamino, (C₃-C₈)-cycloalkylcarbonyl-N-(C₁-C₁₀)-alkylamino, (C₆-C₁₂)-arylcarbonyl-N-(C₁-C₁₀)-aklylamino, (C₇-C₁₁)-aralkylcarbonyl-N-(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino(C₁-C₈)-alkyl, amino-(C₁-C₁₀)-alkyl, N-(C₁-C₁₀) alkylamino(C₁-C₁₀)-alkyl, N,N-di(C₁-C₁₀)-alkylamino-(C₁-C₁₀)-alkyl, (C₃-C₈)cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N-(C₁-C₁₀)-alkylsulfamoyl, N,N-di(C₁-C₁₀)-alkylsulfamoyl, (C₃-C₈)-cycloalkylsulfamoyl, N-(C₆-C₁₂)-alkylsulfamoyl, N-(C₇-C₁₆)-aralkylsulfamoyl, N-(C₁-C₁₀)-alkyl-N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, N-((C₁-C₁₀)-alkyl)-(C₁-C₁₀)-alkylsulfonamido, (C₇-C₁₆)-aralkylsulfonamido, or N-((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)alkyl, (C₁-C₁₂)-alkoxy-(C₁ C₁₂)-alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkyl-carbonyl, (C₆-C₁₂)-arylcarbonyl, (C₇-C₁₆) aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenycarbonyloxy, (C₂-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N-(C₆-C₁₂)-arylcarbamoyl, N-(C₇-C₁₆)-aralkylcarbamoyl, N-(C₁-C₁₀)-alkyl-N-(C₆-C₁₂)-arylcarbamoyl, N-(C₁-C₁₀)-alkyl-N-(C₇-C₁₆)-aralkylcarbamoyl, N-((C₁-C₁₀)-alkoxy-(C₁-C₁₀)-alkyl)-carbamoyl, N-((C₆-C₁₂)-aryloxy-(C₁-C₁₀)-alkyl)-carbamoyl, N-((C₇-C₁₆)-aralkyloxy-(C₁-C₁₀)-alkyl)-carbamoyl, N-(C₁-C₁₀)-alkyl-N-((C₁-C₁₀)-alkoxy-(C₁-C₁₀)-alkyl)-carbamoyl, N-(C₁-C₁₀)-alkyl-N-((C₆-C₁₂)-aryloxy-(C₁-C₁₀)-alkyl)-carbamoyl, N-(C₁-C₁₀)-alkyl-N-((C₇-C₁₆)-aralkyloxy-(C₁-C₁₀)-alkyl)-carbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N(C_{1∂-C}₁₆)-aralkylcarbamoyloxy, N-(C₁-C₁₀)-alkyl-N-(C₆-C₁₂)-arylcarbamoyloxy, N(C₁-C₁₀)-alkyl-N-(C₇-C₁₆)-aralkylcarbamoyloxy, N-((C₁-C₁₀)-alkyl-carbamoyloxy, N-((C₆-C₁₂)-aryloxy-(C₁-C₁₀)-alkyl)-carbamoyloxy, N-((C₇-C₁₆)-aralkyloxy-(C₁-C₁₀)-alkyl)-carbamoyloxy, N-(C₁-C₁₀)-alkyl-N-((C₁-C₁₀)-alkoxy-(C₁-C₁₀)-alkyl)-carbamoyloxy, N-(C₁-C₁₀)-alkyl-N-((C₆-C₁₂)-aryloxy-(C₁-C₁₀)-alkyl-carbamoyloxy, N-(C₁-C₁₀)-alkyl-N-((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkylaralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N-(C₁-C₁₀)-alkylamino, (C₃-C₈)-cycloalkylcarbonyl-N-(C₁-C₁₀)-alkylamino, (C₆-C₁₂)-arylcarbonyl-N-(C₁-C₁₀)-alkylamino, (C₇-C₁₁)-aralkylcarbonyl-N-(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N-(C₁-C₁₀)-alkylamino-(C₁-C₁₀)alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, or (C₇-C₁₆)-aralkylsulfonyl;
X is O or S:
Q is O, S, NR′, or a bond;
where, if Q is a bond, R⁴ is halogen, nitrile, or trifluoromethyl;
   or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓ—C_{f}H_{(2f+1-g)}—F_{g}, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁₁), aralkyl radical, or a radical of the Formula Z

—[CH₂]ᵥ—[O]_{w}—[CH₂]_{f}—E (Z)
where
E is a heteroaryl radical, a (C₃-C₈)-cycloalkyl radical, or a phenyl radical of the Formula F

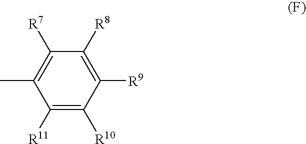

v is 0-6,
w is 0 or 1,
t is 0-3, and
R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—-CF₂-CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N-(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N-(C₃-C₈)-cycloalkylcarbamoyl, N-(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{y} and R^{z} are independently selected from hydrogen (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)aralkoxy, (C₁-C₁₂)-alkylcarbonyl, (C₃-C₈)-cycloalkylcarbonyl, (C₆-C₁₂) arylcarbonyl, (C₇-C₁₆)-aralkylcarbonyl; or further wherein R^{y} and R^{z} together are —[CH2]ₕ, in which a CH₂ group can be replaced by O, S, N-(C₁-C₈)-alkylcarbonylimino, or N-(C₁-C₄)-alkoxycarbonylimino; phenylmercapto, phenylsulfonyl, phenylsulfinyl, sulfamoyl, N-(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are a chain selected from —[CH₂]ₙ—or —CH═CH—CH═CH—, where a CH₂ group of the chain is optionally replaced by O, S, SO, SO₂, or NR^{y}; and n is 3, 4, or 5; and if E is a heteroaryl radical, said radical can carry 1-3 substituents selected from those defined for R⁷—R¹¹, or if E is a cycloalkyl radical, the radical can carry one substituent selected from those defined for R⁷—R¹¹;
or where, if Q is NR′, R⁴ is alternatively R″, where R′ and R″ are identical or different are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl (C₆-C₁₂)-aryloxy-)(C₁-C₈)-alkyl, (C₁-C₁₀)-alkylcarbonyl, optionally substituted (C₇-C₁₆)-aralkylcarbonyl, or optionally substituted C₆-C₁₂)-arylcarbonyl; or R′ and R″ together are —[CH₂]ₕ, in which a CH₂ group can be replaced by O, S, N-acylimino, or N-(C₁-C₁₀)-alkoxycarbonylimino, and h is 3 to 7.
Y is N or CR³;
R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy, (C₁-C₁₂)-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]ₓCfH_{(2f+1-g)}F_{g}, —OCF₂Cl, —OCR₂CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-((C₃-C₈)-cycloalkylcarbamoyl, N-((C₃-C₈)-cycloalkyl-(C₁-C₆)-alkyl-carbamoyl, N-(C₁-C₆)-alkyl-N-((C₃-C₈)-cycloalkyl-(C₁-C₆)-alkyl)-carbamoyl, N-(+)-dehydroabietylcarbamoyl, N-(C₁-C₆)-alkyl-N-(+)-dehydroabietylcarbamoyl, N-(C₆-C₁₂)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, N-((C₁-C₁₈)-alkoxy, (C₁-C₁₀)-alkyl)-carbamoyl, N-((C₆-C₁₆)-aryloxy-(C₁-C₁₀)-alkyl)-carbamoyl, N-((C₇-C₁₆)-aralkyloxy-(C₁-C₁₀)-alkyl)-carbamoyl, N-(C₁-C₁₀)-alkyl-N-((C₁-C₁₀)-alkoxy-(C₁-C₁₀)-alkyl)-carbamoyl, N-(C₁-C₁₀)-alkyl-N-((C₆-C₁₂)-aryloxy-(C₁-C₁₀)-alkyl)-carbamoyl, N-(C₁-C₁₀)-alkyl-N-((C₇-C₁₆)-aralkyloxy-(C₁-C₁₀)-alkyl)-carbamoyl; CON(CH₂)ₕ, in which a CH₂ group can be replaced by OS, S, N-(C₁-C₈)-alkylimino, N-(C₃-C₈)-cycloalkylimino, N-(C₃-C₈)-cycloalkyl-(C₁-C₄)-alkylimino, N-(C₆-C₁₂)-arylimino, N-(C₇-C₁₆)-aralkylimino, N-(C₁-C₄)-alkoxy-(C₁-C₆)-alkylimino, and h is from 3 to 7; a carbamoyl radical of the Formula R.

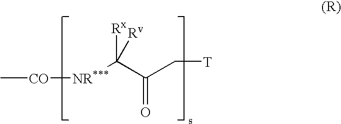

R^{x} and R^{V} are each independently selected from hydrogen, (C₁-C₆)-alkyl, (C₃-C₇)-cycloalkyl, aryl, or the substituent of an α-carbon of an α-amino acid, to which the L—and D-amino acids belong,
s is 1-5,
T is OH, or NR^{*}R^{**}, and R^{*}, R^{**} and R^{***} are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R^{*} and R^{**} together are —[CH₂]ₕ, in which a CH₂ group can be replaced by O, S, SO, SO₂, N-acylamino, N-(C₁-C₁₀)-alkoxycarbonylimino, N-(C₁-C₈)-alkylimino, N-(C₃-C₈)-cycloalkylimino, N-(C₃-C₈)-cycloalkyl-(C₁-C₄)-alkylimino, N-(C₆-C₁₂)-arylimino, N-(C₇-C₁₆)-aralkylimino, N-(C₁-C₈)-alkoxy-(C₁-C₆)-alkylimino, and h is from 3 to 7;
carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-(C₇-C₁₆)-aralkylcarbamoyloxy, N-((C₁-C₁₀)-carbamoyloxy, N-((C₆-C₁₂)-aryloxy-(C₁-C₁₀)-alkyl)-carbamoyloxy, N-((C₇-C₁₆)-aralkyloxy-(C₁-C₁₀)-alkyl-carbamoyloxy, N-(C₁-C₁₀)-alkyl-N-((C₁-C₁₀)-alkoxy-(C₁-C₁₀)-alkyl)-carbamoyloxy, N-(C₁-C₁₀)-alkyl-N-((C₆-C₁₂)-aryloxy-(C₁-C₁₀)-alkyl)-carbamoyloxy, N-(C₁-C₁₀)-alkyl-N-((C₇-C₁₆)-aralkyloxy-(C₁-C₁₀)-alkyl)-carbamoyloxyamino, (C₁-C₁₂)-alkylamino, di-(C₁-C₁₂)-alkyamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkyl-aralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N-(C₁-C₁₀)-alkylamino, (C₃-C₈)-cycloalkanoyl-N-(C₁-C₁₀)-allylamino, (C₆-C₁₂)-aroyl-N-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N-(C₁-C₁₀)-alkylamino-(C₁-C₁₀)-alkyl, N,N-di(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfonyl-(C₁-C₆)-alkyl,(C₆-C₁₂)-arylmercapto-(C₁-C₆)-alkyl, (C₆-₁₂)-arylsulfinyl--(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁C₆)-alkyl, (C₇-C₁₆)-aralkylsulfonyl-(C₁-C₆)-alkyl, sulfamoyl, N-(C₁-C₁₀)-alkylsulfamoyl, N,N-di-(C₁-C₁₀)-alkylsulfamoyl, (C₃-C₈)-cycloalkylsulfamoyl, N-(C₆-C₁₂)-arylsulflamoyl, N-(C₇-C₁₆)-aralkylsulfamoyl, N-(C₁-C₁₀)-alkyl-N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, N-((C₁-C₁₀)-alkyl)-(C₁-C₁₀)-alkylsulonamido, (C₇-C₁₆)-aralkylsulfonamido, and N-((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₂)-alkoxy(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₁-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂ )-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di(C₁-C₁₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-(C₃-C₈)-cycloalkylcarbamoyl, N-((C₃-C₈)-cycloalkyl-(C₁-C₆)-alkyl)carbamoyl, N-(C₁-C₆)-alkyl-N-((C₃-C₈)-cycloalkyl-(C₁-C₆)-alkyl)carbamoyl, N-(+)-dehydroabietylcarbamoyl, N-(C₁-C₆)-alkyl-N-(+)-dehydroabietylcarbamoyl, N-(C₆-C₁₂)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, N-((C₁-C₁₆)-alkoxy-(C₁-C₁₆)-aralkyloxy-(C₁-C₁₀)-alkyl)carbamoyl, N-(C₁-C₁₀)-alkyl-((C₁-C₁₀)-alkoxy-(C₁-C₁₀)-alkyl)carbamoyl, N-(C₁-C₁₀)-alkyl-N-((C₆-C₁₂)-aryloxy-(C₁-C₁₀)-alkyl)carbamoyl, N-(C₁-C₁₀)-alkyl-N-((C₇-C₁₆)-aralkyloxy-(C₁-C₁₀)-alkyl)-carbamoyl, CON(CH₂)ₕ, in which a CH₂ group can be replaced by, O, S, N-(C₁-C₈)-alkylimino, N-(C₃-C₈)-cycloalkylimino, N-(C₃-C₈)-cycloalkyl-(C₁-C₄)-alkylimino, N-(C₆-C₁₂)-arylimino, N-(C₇-C₁₆)-aralkylimino, N-(C₁-C₄)-alkoxy-(C₁-C₆)-alkylimino, and h is from 3 to 7; carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₆)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-(C₇-C₁₆)-aralkylcarbamoyloxy, N-((C₁-C₁₀)-alkyl)carbamoyloxy, N-((C₆-C₁₂)-aryloxy-(C₁-C₁₀)-alkyl)carbamoyloxy, N-((C₇-C₁₆)-aralkyloxy-(C₁-C₁₀)-alkyl)carbamoyloxy, N-(C₁-C₁₀)-alkyl-N-(C₁-C₁₀)-alkoxy-(C₁-C₁₀)-alkyl)carbamoyloxy, N-(C₁-C₁₀)-alkyl-N-((C₆-C₁₂)-aryloxy-(C₁-C₁₀)-alkyl)carbamoyloxy, N-(C₁-C₁₀)-alkyl-N-((C₇-C₁₆)-aralkyloxy-(C₁-C₁₀)-alkyl)carbamoyloxy, amino, (C₁-C₁₂)-alkenylamino, di-(C₁-C₁₂)-alkynylamino, (C₃-C₈)-cycloalkylamino, (C₃-C₁₂)-aroylamino, (C₇-C₁₆)-aralkanoylamino, (C₁-C₁₂)-alkanoyl-N-(C₁-C₁₀)-alkylamino, (C₃-C₈)-cycloalkanoyl-N-(C₁-C₁₀)-alkylamino, (C₆-C₁₂)-aroyl-N-(C₁-C₁₀)-alkylamino, (C₇-C₁₁)-aralkanoyl-N-(C₁-C₁₀)-alkylamino, (C₁-C₁₂)-alkanoylamino-(C₁-C₈)-alkyl, (C₃-C₈)-cycloalkanoylamino-(C₁-C₈)-alkyl, amino-(C₆-C₁₂)-aroylamino-(C₁-C₈)-alkyl, (C₇-C₁₆)-aralkanoylamino-(C₁-C₈)-alkyl, amino-(C₁-C₁₀)-alkyl, N-(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfinyl, (C₁-C₁₂)-(C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;
or wherein R¹ and R², or R² and R³ form a chain [CH₂]ₒ, which is saturated or unsaturated by a C═C double bone, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4, or 5;
or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;
or wherein R¹ and R², or R² and R³ form a carbocyclic or heterocyclic 5- or 6-membered aromatic ring;
or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the Formulae Ia, Ib and Ic:

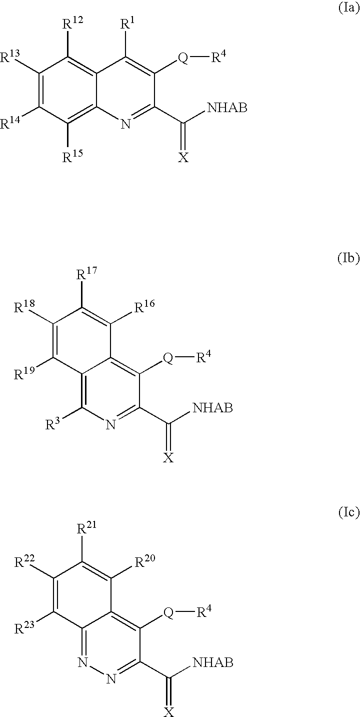

and the substituents R¹² to R²³ in each case independently of each other have the meaning of R¹, R² and R³;
or wherein the radicals R¹ and R², together with the pyridine carrying them, form a compound of Formula Id:

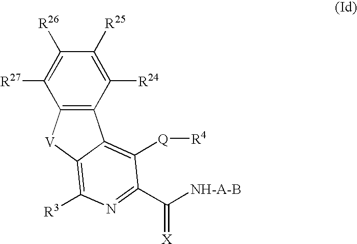

where
V is S, O, or NR^{k}, and R^{k} is selected from hydrogen, (C₁-C₆)-alkyl, aryl, or benzyl; where an aryl radical may be optionally substituted by 1 to 5 substituents as defined above; and
R²⁴, R²⁵, R²⁶, and R²⁷ in each case independently of each other have the meaning of R¹, R² and R³;
f is 1 to 8;
g is 0 or 1 to (2f+1);
x is 0 to 3; and
h is 3 to 7;
including the physiologically active salts, esters, and prodrugs derived therefrom.

Exemplary compounds according to Formula I are described in European Patent Nos. EP0650960 and EP0650961. All compounds listed in EP0650960 and EP0650961, in particular, those listed in the compound claims and the final products of the working examples, are hereby incorporated into the present application by reference herein. Additionally, exemplary compounds according to Formula I are described in U.S. Pat. No. 5,658,933. All compounds listed in U.S. Pat. No. 5,658,933, in particular, those listed in the compound claims and the final products of the working examples, are hereby incorporated into the present application by reference herein.

Additional compounds according to Formula I are substituted heterocyclic carboxyamides described in U.S. Pat. No. 5,620,995; 3-hydroxypyridine-2-carboxamidoesters described in U.S. Pat. No. 6,020,350; sulfonamidocarbonylpyridine-2-carboxamides described in U.S. Pat. No. 5,607,954; and sulfonamidocarbonyl-pyridine-2-carboxamides and sulfonamidocarbonyl-pyridine-2-carboxamide esters described in U.S. Pat. No. 5,610,172 and 5,620,996. All compounds listed in these patents, in particular, those compounds listed in the compound claims and the final products of the working examples, are hereby incorporated into the present application by reference herein.

Exemplary compounds according to Formula Ia are described in U.S. Pat. Nos. 5,719,164 and 5,726,305. All compounds listed in the foregoing patents, in particular, those listed in the compound claims and the final products of the working examples, are hereby incorporated into the present application by reference herein. Exemplary compounds according to Formula Ib are described in U.S. Pat. No. 6,093,730. All compounds listed in U.S. Pat. No. 6,093,730, in particular, those listed in the compound claims and the final products of the working examples, are hereby incorporated into the present application by reference herein

In certain embodiments, compounds of the invention are pyridine-2-carboxamides. In one embodiment, the compound is selected from a compound of the Formula I, wherein
A is —CR⁵R⁶—, and R⁵ and R⁶ are each independently selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂—G carboxyl radical, where G is a radical of an alcohol G—OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl, radical, (C₄-C₂₀)-alkenynyl radical;
X is O;
Q is O;
R⁴ is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
Y is CR^{3.}; R¹, R² and R³ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCR₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-(C₃-C₈)-cycloalkylcarbamoyl, N-((C₃-C₈)-cycloalkyl-(C₁-C₆)-alkyl)carbamoyl, N-(C₁-C₆)-alkyl-N-((C₃-C₈)-cycloalkyl-(C₁-C₆)-alkyl)carbamoyl, N-(+)-dehydroabietylcarbamoyl, N-(C₁-C₆)-alkyl-N-(+)-dehydroabietylcarbamoyl, N-(C₆-C₁₂)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, N-((C₁-C₁₆)-aralkyloxy-(C₁-C₁₀)-alkyl)carbamoyl, N-((C₆-C₁₆)-aryloxy-(C₁-C-₁₀)-alkoxy-(C₁-C₁₀)-alkyl)carbamoyl, N-(C₇-C₁₆)-aralkyloxy-(C₁-C₁₀)-alkyl)-carbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-(C₇-C₁₆)-aralkylcarbamoyloxy, N-((C₁-C₁₀)-alkyl-carbamoyloxy, N-(C₁-C₁₀)-alkyl-N-((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkyl-aralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-aroyl-N-(C₁-C₁₀)-alkylamino, (C₇-C₁₁)-aralkanoyl-N-(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N-(C₁-C₁₀)-alkylsulfamoyl, N,N-di-(C₁-C₁₀)-alkylsulfamoyl, (C₃-C₈)-cycloalkylsulfamoyl, N-(C₆-C₁₂)-arylsulfamoyl, N-(C₇-C₁₆)-aralkylsulfamoyl, N-(C₁-C₁₀)-alkyl-N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, (C₇-C₁₆)-aralkylsulfonamido, and N-((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂Cl, and —OCF₂—CHFCl;
   x is 0 to 3;
   f is 1 to 8; and
   g is 0 or 1 to (2f+1);
   including the physiologically active salts, esters, and prodrugs derived therefrom.

Pyridine-2-carboxamides of Formula I include, but are not limited to, [(3-methoxy-pyridine-2-carbonyl)-amino]-acetic acid, 3-methoxypyridine-2-carboxylic acid N-(((hexadecyloxy)-carbonyl)-methyl)-amide hydrochloride, 3-methoxypyridine-2-carboxylic acid N-(((1-octyloxy)-carbonyl)-methyl)-amide, 3-methoxypyridine-2-carboxylic acid N-(((hexyloxy)-carbonyl)-methyl)-amide, 3-methoxypyridine-2-carboxylic acid N-(((butyloxy)-carbonyl)-methyl)-amide, 3-methoxypyridine-2-carboxylic acid N-(((2-nonyloxy)-carbonyl)-methyl)-amide racemate, 3-methoxypyridine-2-carboxylic acid N-(((heptyloxy)-carbonyl)-methyl)-amide, 3-benzyloxypyridine-2-carboxylic acid N-(((octyloxy)-carbonyl)-methyl)-amide, 3-benzyloxypyridine-2-carboxylic acid N-(((butyloxy)-carbonyl)-methyl)-amide, 5-(((3-(1-butyloxy)-propyl)-amino)-carbonyl)-3-methoxypyridine-2-carboxylic acid N-(((benzyloxycarbonyl)-methyl)-amide, 5-(((3-1-butyloxy)-propyl)-amino)-carbonyl)-3-methoxypyridine-2-carboxylic acid N-(((1-butyloxy)-carbonyl)-methyl)-amide, 5(((3-lauryloxy)-propyl)amino-carbonyl)-3-methoxypyridine-2-carboxylic acid N-(((benzyloxy)-carbonyl)-methyl)-amide, [(3-hydroxy-pyridine-2-carbonyl)-amino]-acetic acid, and [(3-methoxy-pyridine-2-carbonyl)-amino]-acetic acid.

In certain embodiments, compounds of the invention are quinoline-2-carboxamides. In one embodiment, the compound is selected from a compound of the Formula Ia wherein
A is —CR⁵R⁶—, and R⁵ and R⁶ are each independently selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂—G carboxyl radical, where G is a radical of an alcohol G—OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
X is O;
Q is ):
R⁴ is selected from the group consisting of hydrogen, (C₁-C₁₆)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
R¹, R¹², R¹³, R¹⁴ and R¹⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-arloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-(C₃-C₈)-cycloalkylcarbamoyl, N-((C₃-C₈)-cycloalkyl-(C₁-C₆)-alkyl)-carbamoyl, N-(+)-dehydroabietylcarbamoyl, N-(C₁-C₆)-alkyl-N-(+)-dehydroabietylcarbamoyl, N-(C₆-C₁₂)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-(C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkyl-aralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N-(C₁-C₁₀)-alkylamino, (C₃-C₈)-cycloalkanoyl-N-(C₁-C₁₀)-alkylamino, (C₆-C₁₂)-aroyl-N-(C₁-C₁₀)-alkylamino, (C₇-C₁₁)-aralkanoyl-N-(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N-(C₁-C₁₀)-alkylsulfamoyl, N,N-di-(C₁-C₁₀)-alkylsulfamoyl, (C₃-C₈)-cycloalkylsulfamoyl, N-(C₆-C₁₂)-arylsulfamoyl, N-(C₇-C₁₆)-aralkylsulfamoyl, N-(C₁-C₁₀)-alkyl-N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, (C₇-C₁₆)-aralkylsulfonamido, and N-((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂Cl, and —OCR₂—CHFCl;
x is 0 to 3;
f is 1 to 8; and
g is 0 or 1 to (2f+1);
including the physiologically active salts, esters, and prodrugs derived therefrom.

Quinoline-2-carboxamides of Formula Ia include, but are not limited to, N-((3-Hydroxy-6-isopropoxy-quinoline-2-carbonyl)-amino)-acetic acid, N-((6-(1-butyloxy)-3-hydroxyquinolin-2-yl)-carbonyl)-glycine, [(3-hydroxy-6-trifluoromethoxy-quinoline-2-carbonyl)-amino]-acetic acid, [(7-Chloro-3-hydroxy-quinoline-2-carbonyl)-amino]-acetic acid], and [(6-chloro-3-hydroxy-quinoline-2-carbonyl)-amino]-acetic acid.

In certain embodiments, compounds of the invention are isoquinoline-3-carboxamides. In one embodiment, the compound is selected from a compound of the Formula Ib wherein
A is —CR⁵R⁶—, and R⁵ and R⁶ are each independently selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂—G carboxyl radical, where G is a radical of an alcohol G—OH in which G is selected from the group consisting of (C₁-C₂₀)-alkyl radical, (C₃-C₈)-cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
X is O;
Q is O;
R⁴ is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
R³, R¹⁶, R¹⁷, R¹⁸ and R¹⁹ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-(C₃-C₈)-cycloalkylcarbamoyl, N-((C₃-C₈)-cycloalkyl-(C₁-C₆)-alkyl)-carbamoyl, N-(+)-dehydroabietylcarbamoyl, N-(C₁-C₆)-alkyl-N-(+)-dehydroabietylcarbamoyl, N-(+)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-((C₇-C₁₆)-aralkyloxy-(C₁-C₁₀)-alkyl)-carbamoyloxyamino, (C₁-C₁₂)-alkylamino, di-C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkyl-aralkylamino, N-alkyl-arylamino, (C₁-C₁₂)-arylamino, N-(C₁-C₁₂)-alkoxy-N-(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N-(C₁-C₁₀)-alkylamino, (C₃-C₈)-cycloalkanoyl-N-(C₁-C₁₀)-alkylamino, (C₈-C₁₂)-aroyl-N-(C₁-C₁₀)-alkylamino, (C₇-C₁₁)-aralkanoyl-N-(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N-(C₁-C₁₀)-alkylsulfamoyl, N,N-di-(C₁-C₁₀)-alkylsulfamoyl, (C₃-C₈)-cycloalkylsulfamoyl, N-(C₆-C₁₂)-arylsulfamoyl, N-(C₇-C₁₆)-aralkylsulfamoyl, N-(C₁-C₁₀)-alkyl-N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, (C₇-C₁₆)-aralkylsulfonamido, and N-((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g},—OCF₂Cl, and —OCF₂—CHFCl;
x is 0 to 3;
f is 1 to 8; and
g is 0 or 1 to (2f+1); including the physiologically active salts, esters, and prodrugs derived therefrom.

In another embodiment, compounds of the invention are isoquinoline-3-carboxamides, such as disclosed in WO 2004/108681, represented by Formula Ic

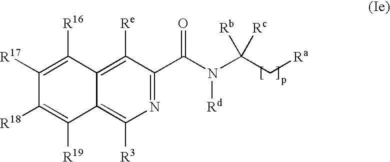

wherein
p is zero or one;
R^{a} is —COOH or —WR⁵⁰; provided that when R^{a} is —COOH then p is zero and when R^{a} is —WR⁵⁰ then p is one;
W is selected from the group consisting of oxygen, —S(O)ₙ— and —NR⁵¹— where n is zero, one or two, R⁵¹ is selected from the group consisting of hydrogen, alkyl, substituted alkyl, acyl, aryl, and R⁵⁰ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁵⁰ and R⁵¹, together with the nitrogen atom to which they are bound, can be joined to form a heterocyclic or a substituted heterocyclic group, provided that when W is —S(O)ₙ— and n is one or two, then R⁵⁰ is not hydrogen;
R³ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶⁰ where X is oxygen, —S(O)ₙ— or —NR⁷⁰— where n is zero, one or two; R⁶⁰ is selected from the group consisting of alkyl, substituted alkyl, aryl, substituted aryl, heteroaryl, substituted heteroaryl, heterocyclic and substituted heterocyclic; and R⁷⁰ is hydrogen, alkyl or aryl; or, when X is —NR⁷⁰—, then R⁶⁰ and R⁷⁰, together with the nitrogen atom to which they are bound, can be joined to form a heterocyclic or substituted heterocyclic group;
R¹⁷ and R¹⁸ are independently selected from the group consisting of hydrogen, alkyl, substituted alkyl, aryl, substituted aryl, heteroaryl, substituted heteroaryl, halo, hydroxy, cyano, —S(O)ₙ— or —N(R⁸⁰)—r⁸⁰ where n is 0, 1, or 2, —NR⁸⁰C(O)NR⁸⁰R⁸⁰, —XR⁸⁰ where X is oxygen, —S(O)ₙ— or —NR⁹⁰ — where n is zero, one or two, each R⁸⁰ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R⁸⁰ is not hydrogen, and R⁹⁰ is selected from the group consisting of hydrogen, alkyl, aryl, or R¹⁷, R¹⁸ together with the carbon atom pendent thereto, form an aryl substituted aryl, heteroaryl, or substituted heteroaryl;
R¹⁶ and R¹⁹ are independently selected from the group consisting of hydrogen, halo, alkyl, substituted alkyl, alkoxy, substituted alkoxy, aryl, substituted aryl, heteroaryl, substituted heteroaryl and —XR⁶⁰ where X is oxygen, —S(O)ₙ— or —NR⁷⁰— where n is zero, one or two, R⁶⁰ is selected from the group consisting of alkyl, substituted alkyl, aryl, substituted aryl, heteroaryl, substituted heteroaryl, heterocyclic an substituted heterocyclic, and R⁷⁰ is hydrogen, alkyl or aryl or, when X is —NR⁷⁰—, then R⁷⁰ and R⁶⁰, together with the nitrogen atom to which they are bound, can be joined to form a heterocyclic or substituted heterocyclic group;
R^{b} is selected from the group consisting of hydrogen, deuterium and methyl;
R^{c} is selected from the group consisting of hydrogen, deuterium, alkyl and substituted alkyl; alternatively, R^{b} and R^{c} and the carbon pendent thereto can be joined to form cycloalkyl, substituted cycloalkyl, heterocyclic or substituted heterocyclic group;
R^{d} is selected from the group consisting of hydrogen and alkyl or R^{d} together with R^{c} and the nitrogen pendent thereto can be joined to form a heterocyclic or substituted heterocyclic group; and
R^{e} is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)ₙ—R⁹⁵ wherein R⁹⁵ is selected from the group consisting of alkyl, substituted alkyl, cycloalkyl, substituted cycloalkyl, aryl, substituted aryl, heteroaryl and substituted heteroaryl and n is zero, one or two;
and pharmaceutically acceptable salts, esters, and prodrugs thereof.

In one embodiment, the compounds of Formula Ic are represented by Formula Ie(i)

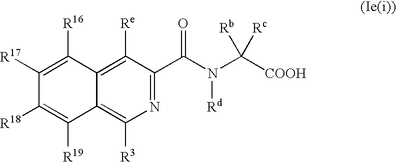

wherein R³R¹⁶, R¹⁷, R¹⁸, R¹⁹, R^{b}, R^{c}, R^{d}, and R³ are as defined above in the discussion for Formula Ie; and pharmaceutically acceptable salts, esters, and prodrugs thereof.

In particular embodiments, the invention is directed to compounds of Formula Ie(i) wherein
R³ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶⁰ where X is oxygen, —S(O)ₙ— or —NR⁷⁰— where n is zero, one or two, R⁶⁰ is selected from the group consisting of alkyl, substituted alkyl, aryl, substituted aryl, heteroaryl, substituted heteroaryl, heterocyclic and substituted heterocyclic, and R⁷⁰ is hydrogen, alkyl or aryl;
R¹⁷ and R¹⁸ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁸⁰ where X is oxygen, —X(O)ₙ— or —NR⁹⁰— where n is zero, one or two, R⁸⁰ is selected from the group consisting of alkyl, substituted alkyl, aryl, substituted aryl, heteroaryl, substituted heteroaryl, heterocyclic and substituted heterocyclic, and R⁹⁰ is hydrogen, alkyl or aryl;
R¹⁶ and R¹⁹ are independently selected from the group consisting of hydrogen, halo, alkyl, substituted alkyl, alkoxy, substituted alkoxy, aryl, substituted aryl, heteroaryl, substituted heteroaryl and —XR⁶⁰ where X is oxygen, —S(O)ₙ— or —NR⁷⁰— where n is zero, one or two, R⁶⁰ is selected from the group consisting of alkyl, substituted alkyl, aryl, substituted aryl, heteroaryl, substituted heteroaryl, heterocyclic and substituted heterocyclic, and R⁷⁰ is hydrogen, alkyl or aryl;
R^{b} is selected from the group consisting of hydrogen and methyl;
R^{c} is selected from the group consisting of alkyl and substituted alkyl; or R^{a} or R^{b} may be joined to form a cycloalkyl, substituted cycloalkyl, heterocyclic or substituted heterocyclic; and
R^{d} is selected from the group consisting of hydrogen and alkyl or R^{d} together with R^{c} and the nitrogen pendent thereto forms a heterocyclic or substituted heterocyclic group; and
R^{e} is hydroxy;
and pharmaceutically acceptable salts, esters, and prodrugs thereof.

In another embodiment, the compounds of Formula Ie are represented by the Formula Ie(ii)

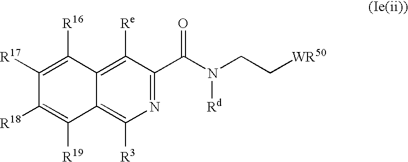

wherein R³, R¹⁶, R¹⁷, R¹⁸, R¹⁹, R^{d}, R^{e}, and WR⁵⁰ are as defined above in the discussion for Formula Ie; and pharmaceutically acceptable salts, esters, and prodrugs thereof.

In particular embodiments, the invention is directed to compounds of Formula Ie(ii) wherein
W is selected from the group consisting of oxygen, —S(O)ₙ— and —NR⁵¹— where n is zero, one or two, R⁵¹ is selected from the group consisting of hydrogen, alkyl, substituted alkyl, acyl, aryl, substituted aryl, heteroaryl, substituted heteroaryl, heterocyclic and substituted heterocyclic;
R⁵⁰ is selected from the group consisting of hydrogen, alkyl, substituted alkyl, aryl, substituted aryl, heteroaryl, substituted heteroaryl, heterocyclic and substituted heterocyclic; R^{d} is selected from hydrogen and alkyl; R^{c}is hydroxy; R¹⁷ and R¹⁸ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁸⁰ where X is oxygen, —S(O)ₙ— or —NR⁹⁰ — where n is zero, one or two, R⁸⁰ is selected from the group consisting of alkyl, substituted alkyl, aryl, substituted aryl, heteroaryl, substituted heteroaryl, heterocyclic and substituted heterocyclic, and R⁹⁰ is hydrogen, alkyl or aryl; and
R¹⁶ and R¹⁹ are independently selected from the group consisting of hydrogen, halo, alkyl, substituted alkyl, alkoxy, substituted alkoxy, aryl, substituted aryl, heteroaryl, substituted heteroaryl and —XR⁶⁰ where X is oxygen, —S(O)ₙ— or —NR⁷⁰— where n is zero, one or two, R⁶⁰ is selected from the group consisting of alkyl, substituted alkyl, aryl, substituted aryl, heteroaryl, substituted heteroaryl, heterocyclic and substituted heterocyclic, and R⁷⁰ is hydrogen, alkyl or aryl;
and pharmaceutically acceptable salts, esters, and prodrugs thereof.

In another embodiment, the compounds of Formula Ie are represented by the Formula Ie(iii)

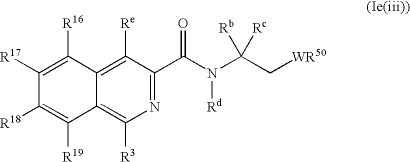

where R³R¹⁶, R¹⁷, R¹⁸, R¹⁹, R^{b}, R^{c}, R^{d}, R^{e}, and WR⁵⁰ are as defined above in the discussion for Formula Ie; and pharmaceutically acceptable salts, esters, and prodrugs thereof.

In particular embodiments, the invention is directed to compounds of Formula Ie(III) wherein
W is selected from the group consisting of hydrogen, alkyl, substituted alkyl, aryl, substituted aryl, heteroaryl, substituted heteroaryl, heterocyclic and substituted heterocyclic;
R⁵⁰ is selected from the group consisting of hydrogen, alkyl, substituted alkyl, aryl, substituted aryl, heteroaryl, substituted heteroaryl, heterocyclic and substituted heterocyclic;
R³ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶⁰ where X is oxygen, —S(O)ₙ— or —NR⁷⁰— where n is zero, one or two, R⁶⁰ is selected from the group consisting of alkyl, substituted alkyl, aryl, substituted aryl, heteroaryl, substituted heteroaryl, heterocyclic and substituted heterocyclic, and R⁷⁰ is hydrogen, alkyl, or aryl;
R¹⁷ and R¹⁸ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁸⁰ where X is oxygen, —S(O)ₙ— or —NR⁹⁰— where n is zero, one or two, R⁸⁰ is selected from the group consisting of alkyl, substituted alkyl, aryl, substituted aryl, heteroaryl, substituted heteroaryl, heterocyclic and substituted heterocyclic, and R⁹⁰ is hydrogen, alkyl, or aryl;
R¹⁶ and R¹⁹ are independently selected from the group consisting of hydrogen, halo, alkyl, substituted alkyl, alkoxy, substituted alkoxy, aryl, substituted aryl, heteroaryl, substituted heteroaryl and —XR⁶⁰ where X is oxygen, —S(O)ₙ— or —NR⁷⁰— where n is zero, one or two, R⁶⁰ is selected from the group consisting of alkyl, substituted alkyl, aryl, substituted aryl, heteroaryl, substituted heteroaryl, heterocyclic and substituted heterocyclic, and R⁷⁰ is hydrogen, alkyl, or aryl;
R^{b} is selected from the group consisting of hydrogen and methyl;
R^{c} is selected from the group consisting of alkyl and substituted alkyl; or R^{b} and R^{c} can be joined to form cycloalkyl, substituted cycloalkyl, heterocyclic or substituted heterocyclic
R^{d} is selected from the group consisting of hydrogen and alkyl or R^{d} together with R^{c} and the nitrogen pendent thereto forms a heterocyclic or substituted heterocyclic group; and
R^{e} is hydroxy;
and pharmaceutically acceptable salts, esters, and prodrugs thereof.

In another embodiment, the compounds of Formula Ie are represented by the Formula Ie(iv)

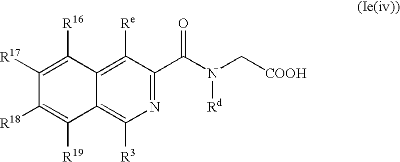

wherein R³, R¹⁶, R¹⁷, R¹⁸, R¹⁹, R^{d}, and R^{e} are as defined above in the discussion for Formula Ie; and pharmaceutically acceptable salts, esters, and prodrugs thereof.

In one particular embodiment, the invention is directed to compounds of Formula Ie(iv) wherein
R^{d} is selected from hydrogen and alkyl;
R^{c} is hydroxy;
R³ is selected from the group consisting of hydrogen, alkyl, substituted alkyl, alkoxy, substituted alkoxy, aryl substituted aryl, halo, heteroaryl, substituted heteroaryl, heterocyclic, substituted heterocyclic, and —XR⁶⁰ where X is oxygen, —S(O)ₙ— or —NR⁷⁰— where n is zero, one or two, R⁶⁰ is selected from the group consisting of alkyl, substituted alkyl, aryl, substituted aryl, heteroaryl, substituted heteroaryl, heterocyclic and substituted heterocyclic, and R⁷⁰ is hydrogen, alkyl or aryl;
R¹⁷ and R¹⁸ are independently selected from the group consisting of hydrogen, alkyl, substituted alkyl, aryl, substituted aryl, heteroaryl, substituted heteroaryl, halo, hydroxy, cyano, —XR⁸⁰ where X is oxygen, —S(O)ₙ— or —NR⁹⁰— where n is zero, one or two, R⁸⁰ is selected from the group consisting of alkyl, substituted alkyl, aryl, substituted aryl, heteroaryl, substituted heteroaryl, heterocyclic and substituted heterocyclic, and R⁹⁰ is hydrogen, alkyl or aryl; and
R¹⁶ and R¹⁹ are independently selected from the group consisting of hydrogen, halo, alkyl, substituted alkyl, alkoxy, substituted alkoxy, aryl, substituted aryl, heteroaryl, substituted heteroaryl and —XR⁶⁰ where X is oxygen, —S(O)ₙ— and —NR⁷⁰— where n is zero, one or two, R⁶⁰ is selected from the group consisting of alkyl, substituted alkyl, aryl, substituted aryl, heteroaryl, substituted heteroaryl, heterocyclic and substituted heterocyclic, and R⁷⁰ is hydrogen, alkyl or aryl;
and pharmaceutically acceptable salts, esters, and prodrugs thereof.

In certain embodiments of compounds of Formula Ie including, but not limited to, certain compounds of Formulae Ie(i), Ie(ii), Ie(iii), and Ie(iv), R³ is selected from the group consisting of hydrogen, alkyl, substituted alkyl, halo, alkoxy, aryloxy, substituted aryloxy, substituted aryl, alkylthio, aminoacyl, aryl, substituted amino, heteroaryl, heteroaryloxy, —S(O)ₙ-aryl, —S(O)ₙ-substituted aryl, —S(O)ₙ-heteroaryl, and —S(O)ₙ-substituted heteroaryl, where n is zero, one or two. In particular embodiments R³ is selected from the group consisting of (3-methoxyphenyl)sulfanyl; (4-chlorophenyl)sulfanyl; (4-methylphenyl)sulfanyl; 2-fluorophenoxy; 2-methoxyphenoxy; (2-methoxyphenyl(sulfanyl 3-fluorophenoxy; 3-methoxyphenoxy; 4-(methylcarbonylamino)phenoxy; 4-(methylsulfonamido)phenoxy; 4-fluorophenoxy; 4-methoxyphenoxy; 4-methoxyphenysulfanyl; 4-methylphenyl; bromo; chloro; dimethylaminomethyl; ethoxy; ethylsulfanyl; hydrogen; isopropyl; methoxy; methoxymethyl; methyl; N,N-dimethylaminocarbonyl; naphth-2-yloxy; naphthylsulfanyl; phenoxy; phenyl; phenylamino; phenylsulfinyl; phenylsulfanyl; pyridin-2-yloxy; pyridin-2-yl; and pyridin-2-ylsulfanyl.

In certain embodiments of compounds of Formula Ie including, but not limited to, certain compounds of Formulae Ie(i), Ie(ii), Ie(iii), and Ie(iv), R¹⁶ is hydrogen or phenyl.

In certain embodiments of compounds of Formula Ie including, but not limited to, certain compounds of Formulae Ie(i), Ie(ii), Ie(iii), and Ie(iv), R¹⁷ is selected from the group consisting of substituted aryloxy, substituted alkoxy, alkoxy, substituted alkyl, alkyl, amino, cycloalkyloxy, hydrogen, halo, aryl, —S(O)ₙ-aryl, —S(O)ₙ-substituted aryl, —S(O)ₙ-heteroaryl, and —X(O)ₙ-substituted heteroaryl, where n is zero, one or two, aminocarbonylamino, and heteroaryloxy. In particular embodiments, R¹⁷ is selected from the group consisting of amino; (4-methyl)phenyl-sulfonylaminophenoxy; 3,4-difluorophenoxy; 3,5-difluorophenoxy; 3-fluoro-5-methoxy-phenoxy; 3-chloro-4-fluorophenoxy 4-CF₃-O-phenoxy; 4-CF₃-phenoxy; 4-chlorophenoxy; 4-fluorophenoxy; 4-(4-fluorophenoxy)phenoxy; 4-methoxyphenoxy; benzyloxy; bromo; butoxy; CF₃; chloro; cyclohexyloxy; hydrogen; iodo; isopropoxy; phenoxy; phenyl; phenylsulfanyl; phenylsulfonyl; phenylsulfinyl; phenylurea; pyridin-1-ylsulfanyl; pyridin3-yloxy; and pyridin-4-ylsulfanyl.

In some embodiments of compounds of Formula Ie including, but not limited to, certain compounds of Formulae Ie(i), Ie(ii), Ie(iii), and Ie(iv), R¹⁸ is selected from the group consisting of substituted amino, aryloxy, substituted aryloxy, alkoxy, substituted alkoxy, halo, hydrogen, alkyl, substituted alkyl, aryl, —S(O)ₙ—aryl, —S(O)ₙ-substituted aryl, —S(O)ₙ-cycloalkyl, where n is zero, one or two, aminocarbonylamino, heteroaryloxy, and cycloalkyloxy. In particular embodiments, R¹⁸ is selected from the group consisting of (4-methoxy)phenylsulfonylamino; 2,6-dimethylphenoxy; 3,4-difluorophenoxy; 3,5-difluorophenoxy; 3-chloro-4-fluorophenoxy; 3-methoxy-4-fluorophenoxy; 3-methoxy-5-fluorophenoxy; 4-(methylsulfonamido)phenoxy; 4(phenylsulfonamido)phenoxy; 4-CF₃-O-phenoxy; 4-CF₃-phenoxy; 4-chlorophenoxy; 4-fluorophenoxy; 4-(4-fluorophenoxy)phenoxy; 4-methoxyphenoxy; 4-nitrophenoxy; benzyloxy; bromo; butoxy; CF₃; chloro; cyclohexyloxy; cyclohexysulfanyl; cyclohexysulfonyl; fluoro; hydrogen; iodo; isopropoxy; methyl; phenoxy; phenyl; phenylsulfanyl; phenylsulfinyl; phenylsulfonyl; phenylurea; pyridin-1-ylsulfanyl; pyridin-3-yloxy; and pyridin-4-ylsulfanyl.

Alternatively, R¹⁷ and R¹⁸, combined with the carbon atoms pendent thereto, are joined to form an aryl group. In a particular embodiment, the aryl group is phenyl.

In certain embodiments of compounds of Formula Ie including, but not limited to, certain compounds of Formulae Ie(i), Ie(ii), Ie(iii), and Ie(iv), R¹⁹ is selected from the group consisting of: substituted arylthio, halo, hydrogen, substituted alkyl and aryl. In particular embodiments, R¹⁹ is selected from the group consisting of 4-chlorophenyl sulfanyl; chloro; hydrogen; methoxymethyl; and phenyl.

In certain embodiments of compounds of Formulae Ie, including but not limited to, certain compounds of Formulae Ie(i) and Ie(iii), R^{b} is selected from the group consisting of hydrogen, deuterium, aryl and alkyl. In particular embodiments, R^{b} is selected from the group consisting of phenyl, hydrogen, deuterium and methyl.

In certain embodiments of compounds of Formula Ie including, but not limited to certain compounds of Formulae Ie(i) and Ie(iii), R^{c} is selected from the group consisting of preferably hydrogen, deuterium, alkyl, substituted alkyl, and substituted amino. In particular embodiments, R^{c} is selected from the group consisting of 4-aminobutyl; 4-hydroxybenzyl; benzyl; carboxymethyl; deuterium, hydroxymethyl; imidazol-4-ylmethyl; isopropyl; methyl; and propyl.

Alternatively, R^{b}, R^{c}, and the carbon atom pendent thereto joint to form a cycloalkyl and more preferably cyclopropyl.

In certain embodiments of compounds of Formula Ie including, but not limited to, certain compounds of Formulae Ie(i) and Ie(iii), R^{d} is hydrogen, alkyl or substituted alkyl. In particular embodiments, R^{d} is hydrogen, methyl or carboxylmethyl (—CH₂C(O)OH). Alternatively, R^{c}, R^{d}, and the carbon atom and nitrogen atom respectively pendent thereto join to form a heterocyclic group and more preferably pyrrolidinyl.

In certain embodiments of compounds of Formula Ie including, but not limited to, certain compounds of Formulae Ie(i), Ie(ii), Ie(iii) and Ie(iv), R^{e} is selected from the group consisting of hydrogen, hydroxy, alkoxy, substituted alkoxy, cycloalkoxy, substituted cycloalkoxy, thiol, acyloxy and aryl. In particular embodiments, R^{e} is selected from the group consisting of hydroxy; benzyloxy; ethoxy; thiol; methoxy; methylcarbonyloxy; and phenyl.

In certain embodiments of compounds of Formulae Ie including, but not limited to, certain compounds of Formulae Ie(ii) and Ie(iii), WR⁵⁰ is selected from the group consisting of amino, substituted amino, aminoacyl, hydroxy, and alkoxy. In particular embodiments, WR⁵⁰ is selected from the group consisting of amino; dimethylamino; hydroxy; methoxy; and methylcarbonylamino.

Isoquinoline-3-carboxamides of Formula Ib and Formula Ie include, but are not limited to, N-((1-chloro-4-hydroxy-7-(2-propyloxy) isoquinolin-3-yl)-carbonyl)-glycine, N-((1-chloro-4-hydroxy-6-(2-propyloxy) isoquinolin-3-yl)-carbonyl)-glycine, N-((1-chloro-4-hydroxy-isoquinoline-3-carbonyl)-amino)-acetic acid (Compound A), [[(1-chloro-4-hydroxy-7-methoxy-isoquinoline-3-carbonyl)-amino]-acetic acid], N-((1-chloro-4-hydroxy-6-methoxyisoquinolin-3-yl)-carbonyl)-glycine, N-((7-butyloxy)-1-chloro-4-hydroxyisoquinolin-3-yl)-carbonyl)-glycine, N-((5-benzyloxy-1-chloro-4-hydroxy-isoquinoline-3-carbonyl)-amino)-acetic acid, ((7-benzyloxy-1-chloro-4-hydroxy-isoquinoline-3-carbonyl)-amino)-acetic acid methyl ester, N-((7-benzyloxy-1-chloro-4-hydroxy-isoquinoline-3-carbonyl)-amino)-acetic acid, N-((8-chloro-4-hydroxyisoquinoline-3-yl)-carbonyl)-glycine, N-((7-butoxy-4-hydroxy-isoquinoline-3carbonyl)-amino)-acetic acid (M), [(1,7-dichloro-4-hydroxy-isoquinoline-3-carbonyl)-amino]-acetic acid, [[(6,7-dichloro-4hydroxy-isoquinoline-3caronyl)-amino]-acetic acid]. {[4-hydroxy-1-(naphthalen-2-yloxy)-isoquinoline-3-carbonyl]-amino}-acetic acid, {[4-hydroxy-1-(4-methoxy-phenoxy)-isoquinoline-3-carbonyl]-amino}-acetic acid, {[4-hydroxy-1-(3-methoxy-phenoxy)-isoquinoline-3-carbonyl]-amino}-acetic acid, {[1-(3-fluoro-phenoxy)-4-hydroxy-isoquinoline-3-carbonyl]-amino}-acetic acid, {[1-(4-fluoro-phenoxy)-4-hydroxy-isoquinoline-3-carbonyl]-amino}-acetic acid, {[1-(2-fluoro-phenoxy)-4-hydroxy-isoquinoline-3-carbonyl]-amino}-acetic acid, {[4-hydroxy-1-(2-methoxy-phenoxy)-isoquinoline-3-carbonyl]-amino)-acetic acid, [(4-hydroxy-1-phenylamino-isoquinoline-3-carbonyl)-amino]-acetic acid, [(1-chloro-4-methoxy-isoquinoline-3-carbonyl)-amino]-acetic acid, [(4-hydroxy-1-phenyl-isoquinoline-3-carbonyl)-amino]-acetic acid, [(4-hydroxy-1-methoxy-isoquinoline-3-carbonyl)-amino]-acetic acid, [(1-ethoxy-4-hydroxy-isoquinoline-3-carbonyl)-amino]-acetic acid, [(4-hydroxy-1-phenyl-isoquinoline-3-carbonyl)-amino]-acetic acid, [(4-hydroxy-1-methyl-isoquinoline-3-carbonyl)-amino]-acetic acid, [(4-hydroxy-1-methoxymethyl-isoquinoline-3-carbonyl)-amino]-acetic acid, [(1-dimethylcarbamoyl-4-hydroxy-isoquinoline-3-carbonyl)-amino]-acetic acid, [(4-hydroxy-1-methyl-6-phenoxy-isoquinoline-3-carbonyl)-amino]-acetic acid, [(4-hydroxy-1-methyl-7-phenoxy-isoquinoline-3-carbonyl)-amino]-acetic acid (Compound D), [(4-benzyloxy-1-methyl-7-phenoxy-isoquinoline-3-carbonyl)-amino]-acetic acid, [(4-ethoxy-1-methyl-7-phenoxy-isoquinoline-3-carbonyl)-amino]-acetic acid, [(1-methoxymethyl-7-phenoxy-isoquinoline-3-carbonyl)-amino]-acetic acid, [(4-hydroxy-1-p-tolyl-isoquinoline-3-carbonyl)-amino]-acetic acid, {[7-(4-fluoro-phenoxy)-4-hydroxy-1-methyl-isoquinoline-3-carbonyl]-amino}-acetic acid, {[1-chloro-4-hydroxy-7-(4-methoxy-phenoxy)-isoquinoline-3-carbonyl]-amino}-acetic acid, {[4-hydroxy-7-(4-methoxy-phenoxy)-isoquinoline-3-carbonyl]-amino}-acetic acid (Compound C), {[1-chloro-4-hydroxy-6-(4-methoxy-phenoxy)-isoquinoline-3-carbonyl]-amino}-acetic acid, {[4-hydroxy-6-(4-methoxy-phenoxy)-isoquinoline-3-carbonyl]-amino}-acetic acid, {[1-chloro-4-hydroxy-7-(4-trifluoromethyl-phenoxy)-isoquinoline-3-carbonyl]-amino}-acetic acid, {[4-hydroxy-7-(4-trifluoromethyl-phenoxy)-isoquinoline-3-carbonyl]-amino}-acetic acid, {[1-chloro-4-hydroxy-6-(4-trifluoromethyl-phenoxy)-isoquinoline-3-carbonyl]-amino}-acetic acid, {[4-hydroxy-6-(4-trifluoromethyl-phenoxy)-isoquinoline-3-carbonyl]-amino}-acetic acid, {[1-chloro-7-(4-fluoro-phenoxy)-4-hydroxy-isoquinoline-3-carbonyl]-amino}-acetic acid, {[7-(4-fluoro-phenoxy)-4-hydroxy-isoquinoline-3-carbonyl]-amino}-acetic acid (Compound E), {[1-chloro-6-(4-fluoro-phenoxy)-4-hydroxy-isoquinoline-3-carbonyl]-amino}-acetic acid, {[6-(4-fluoro-phenoxy)-4hydroxy-isoquinoline-3-carbonyl]-amino}-acetic acid, [(7-benzenesulfinyl-4-hydroxy-isoquinoline-3-carbonyl)-amino]-acetic acid, [(7-benzenesulfonyl-4-hydroxy-isoquinoline-3-carbonyl)-amino]-acetic acid, [(6-benzenesulfinyl-4-hydroxy-isoquinoline-3-carbonyl)-amino]-acetic acid, [(6-benzenesulfonyl-4-hydroxy-isoquinoline-3-carbonyl)-amino]-acetic acid, {[4-hydroxy-7-(4-methoxy-benzenesulfonylamino)-isoquinoline-3-carbonyl]-amino}-acetic acid, [(4-hydroxy-1-phenylsulfanyl-isoquinoline-3-carbonyl)-amino-acetic acid, {[1-(4-chloro-phenylsulfanyl)-4-hydroxy-isoquinoline-3-carbonyl]-amino}-acetic acid, [(4-hydroxy-1-p-tolysulfanyl-isoquinoline-3-carbonyl)-amino]-acetic acid, {[4-hydroxy-1-(3-methoxy-phenylsulfanyl)-isoquinoline-3-carbonyl]-amino}-acetic acid, {[4-hydroxy-1-(2-methoxy-phenylsulfanyl)-isoquinoline-3-carbonyl]-amino}-acetic acid, {[4-hydroxy-1-(naphthalen-2-ylsulfanyl)-isoquinoline-3-carbonyl]-amino}-acetic acid, [(1-benzenesulfinyl-4-hydroxy-isoquinoline-3-carbonyl)-amino]-acetic acid, [(1-benzenesulfonyl-4-hydroxy-isoquinoline-3-carbonyl)-amino]-acetic acid, [(1-chloro-4-hydroxy-6,7-diphenoxy-isoquinoline-3-carbonyl)-amino]-acetic acid, [[(4-Hydroxy-1,7-diphenoxy-isoquinoline-3-carbonyl)-amino]-acetic acid], [(4-hydroxy-6,7-diphenoxy-isoquinoline-3-carbonyl)-amino]-acetic acid, {[4-hydroxy-7-(4-nitro-phenoxy)-isoquinoline-3-carbonyl]-amino}-acetic acid, [(4-mercapto-7-phenoxy-isoquinoline-3-carbonyl)-amino]-acetic acid, [(4-mercapto-7-trifluoromethyl-isoquinoline-3-carbonyl)-amino]-acetic acid, {[7-(4-chloro-phenoxy)-4-hydroxy-isoquinoline-3-carbonyl]-amino}-acetic acid, {[6-(4-chloro-phenoxy)-4-hydroxy-isoquinoline-3-carbonyl]-amino}-acetic acid, {[6-(3-fluoro-5-methoxy-phenoxy)-4-hydroxy-isoquinoline-3-carbonyl]-amino}-acetic acid, {[7-(3-fluoro-5-methoxy-phenoxy)-4-hydroxy-isoquinoline-3-carbonyl]-amino}-acetic acid, {[7-(3,4-difluoro-phenoxy)-4-hydroxy-isoquinoline-3-carbonyl]-amino}-acetic acid, {[6-(3,4-difluoro-phenoxy)-4-hydroxy-isoquinoline-3-carbonyl[-amino}-acetic acid, {[hydroxy-7-(4-trifluoromethoxy-phenoxy)-isoquinoline-3-carbonyl]-amino}-acetic acid, {[hydroxy-6-(4-trifluoromethoxy-phenoxy)-isoquinoline-4-carbonyl]amino}-acetic acid, 2-(S)-{[7-chloro-phenoxy)-4-hydroxy-isoquinoline-3-carbonyl]-amino}-propionic acid, 2-(S)-{[6-(4-chloro-phenoxy)-4-hydroxy-isoquinoline-3-carbonyl]-amino}-propionic acid, 2-{[7-(3,4-difluoro-phenoxy)-4-hydroxy-isoquinoline-3-carbonyl]-amino}-propionic acid, 2-(S)-[(4-hydroxy-7-phenylsulfanyl-isoquinoline-3-carbonyl)-amino]-propionic acid, 2-(R)-[(4-hydroxy-7-phenylsulfanyl-isoquinoline-3-carbonyl)-amino]-propionic acid, 2-(R)-[(4-hydroxy-7-phenoxy-isoquinoline-3-carbonyl)-amino]-propionic acid, (S)-2-[(4-Hydroxy-7-phenoxy-isoquinoline-3-carbonyl)-amino]-propionic acid (Compound B), 2-(S)-{[4-hydroxy-7-(methoxy-phenoxy)-isoquinoline-3-carbonyl]-amino}-propionic acid, 2-(S)-[(7-benzenesulfonyl-4-hydroxy-isoquinoline-3-carbonyl)-amino]-propionic acid, (R)-2-[(4-hydroxy-1-methoxymethyl-7-phenoxy-isoquinoline-3-carbonyl)-amino]-propionic acid, (S)-2-[(4-hydroxy-1-methoxymethyl-7-phenoxy-isoquinoline-3-carbonyl)-amino]-propionic acid, (S)-2-[(4-mercapto-7-phenoxy-isoquinoline-3-carbonyl)-amino]-propionic acid, (S)-2-{[1-(4-chloro-phenylsulfanyl)-4-hydroxy-isoquinoline-3-carbonyl)-amino}-propionic acid, (R)-2-{[1-(4-chloro-phenylsulfanyl)-4-hydroxy-isoquinoline-3-carbonyl;]-amino}-propionic acid, [(4-hydroxy-7-phenylsulfanyl-isoquinoline-3-carbonyl)-amino]-acetic, [(4-hydroxy-6-phenylsulfanyl-isoquinoline-3-carbonyl)-amino]-acetic acid, [(1-chloro-4-hydroxy-7-phenylsulfanyl-isoquinoline-3-carbonyl)-amino]-acetic acid, [(1-chloro-4-hydroxy-6-phenylsulfanyl-isoquinoline-3-carbonyl)-amino]-acetic acid, [(1-bromo-4-hydroxy-7-phenylsulfanyl-isoquinoline-3-carbonyl)-amino]-acetic acid, [(1-bromo-4-hydroxy-6-phenylsulfanyl-isoquinoline-3-carbonyl)-amino]-acetic acid, [(4-hydroxy-7-phenoxy-isoquinoline-3carbonyl)-amino]-acetic acid, [(4-hydroxy-6-phenoxy-isoquinoline-3-carbonyl)-amino]-acetic acid, [(1-chloro-4hydroxy-7-phenoxy-isoquinoline-3-carbonyl)-amino]-acetic acid, [(chloro-4-hydroxy-6-phenoxy-isoquinoline-3-carbonyl)-amino]-acetic acid, [(1-bromo-4-hydroxy-7-phenoxy-isoquinoline-3-carbonyl)-amino]-acetic acid, [(1-bromo-4-hydroxy-6-phenoxy-isoquinoline-3-carbonyl)-amino]-acetic acid, {[7-(2,6-dimethyl-phenoxy)-4-hydroxy-isoquinoline-3-carbonyl]-amino}-acetic acid, {[1-chloro-7-(2,6-dimethyl-phenoxy-4-hydroxy-isoquinoline-3-carbonyl]-amino}-acetic acid, {[1-bromo-7-(2,6-dimethyl-phenoxy)-4-hydroxy-isoquinoline-3-carbonyl]-amino}-acetic acid, [(1-bromo-7-chloro-4hydroxy-isoquinoline-3-carbonyl)-amino]-acetic acid, [(1-bromo-6-chloro-4-hydroxy-isoquinoline-3-carbonyl)-amino -acetic acid, [(1-bromo-4-hydroxy-7-trifluoromethyl-isoquinoline-3-carbonyl)-amino]-acetic acid, [(1-bromo-4-hydroxy-6-trifluoromethyl-isoquinoline-3-carbonyl)-amino]-acetic acid, [(4-hydroxy-1-phenoxy-isoquinoline-3-carbonyl)-amino]-acetic acid, [(1,7-dibromo-4-hydroxy-isoquinoline-3-carbonyl)-amino]-acetic acid, [(7-bromo-1-chloro-4-hydroxy-isoquinoline-3-carbonyl)-amino]-acetic acid, [(6-bromo-4-hydroxy-isoquinoline-3-carbonyl)-amino]-acetic acid, [(1-bromo-7-fluoro-4-hydroxy-isoquinoline-3-carbonyl)-amino]-acetic acid, [(7-fluoro-4-hydroxy-isoquinoline-3-carbonyl)-amino]-acetic acid, [(1-chloro-7-fluoro-4-hydroxy-isoquinoline-3-carbonyl)-amino]-acetic acid, [(1-bromo-4-hydroxy-isoquinoline-3-carbonyl)-amino]-acetic acid, [(4-hydroxy-6-phenyl-isoquinoline-3-carbonyl)-amino -acetic acid, [(4-hydroxy-7-phenyl-isoquinoline-3-carbonyl)-amino]-acetic acid, [(1-chloro-4-hydroxy-6-phenyl-isoquinoline-3-carbonyl)-amino]-acetic acid, [(1-chloro-4-hydroxy-7-phenyl-isoquinoline-3-carbonyl)-amino]-acetic acid, [(1-bromo-4-hydroxy-6-phenyl-isoquinoline-3-carbonyl)-amino]-acetic acid, [(1-bromo-4-hydroxy-7-phenyl-isoquinoline-3-carbonyl)-amino]-acetic acid, [(4-hydroxy-5-phenyl-isoquinoline-3-carbonyl)-amino]-acetic acid, [(4-hydroxy-8-phenyl-isoquinoline-3-carbonyl)-amino]-acetic acid, [(1-chloro-4-hydroxy-5-phenyl-isoquinoline-3-carbonyl)-amino]-acetic acid, [(1-chloro-4-hydroxy-8-phenyl-isoquinoline-3-carbonyl)-amino]-acetic acid, [(1-bromo-4-hydroxy-5-phenyl-isoquinoline-3-carbonyl)-amino]-acetic acid, [(1-bromo-4-hydroxy-8-phenyl-isoquinoline-3-carbonyl)-amino]-acetic acid, [(1-ethylsulfanyl-4-hydroxy-isoquinoline-3-carbonyl)-amino]-acetic acid, {[4-hydroxy-1-(4-methoxy-phenylsulfanyl)-isoquinoline-3-carbonyl]-amino)-acetic acid, [(1-chloro-4-hydroxy-7-iodo-isoquinoline-3-carbonyl)-amino]-acetic acid, [(1-chloro-4-hydroxy-6-iodo-isoquinoline-3-carbonyl)-amino]-acetic acid, [(4-hydroxy-7-iodo-isoquinoline-3-carbonyl)-amino]-acetic acid, ](1-bromo-4-hydroxy-7-methyl-isoquinoline-3-carbonyl)-amino]-acetic acid, [(1-bromo-7-butoxy-4-hydroxy-isoquinoline-3-carbonyl)-amino]-acetic acid, [(1-bromo-6-butoxy-4-hydroxy-isoquinoline-3-carbonyl)-amino]-acetic acid, (carboxymethyl-(1-chloro-4-hydroxy-isoquinoline-3-carbonyl)-amino]-acetic acid; [carboxymethyl-(1-chloro-4-hydroxy-6-isopropoxy-isoquinoline-3-carbonyl)-amino]-acetic acid; 1-chloro-4-hydroxy-isoquinoline-3-carboxylic acid (2-amino-ethyl)-amide (trifluoro-acetic acid salt); 1-chloro-4-hydroxy-isoquinoline-3-carboxylic acid (2-methoxy-ethyl)-amide; 1-chloro-4-hydroxy-isoquinoline-3-carboxylic acid )2-hydroxy-ethyl)-amide; 1-chloro-4-hydroxy-isoquinoline-3-carboxylic acid (2-dimethylamino-ethyl)-amide; 1-chloro-4-hydroxy-isoquinoline-3-carboxylic acid (2-acetylamino-ethyl)-amide; 1-chloro-4-hydroxy-6-isopropoxy-isoquinoline-3-carboxylic acid (2-hydroxy-ethyl)-amide; 1-chloro-4-hydroxy-6-isopropoxy-isoquinoline-3-carboxylic acid (2-methoxy-ethyl)-amide; 1-chloro-4-hydroxy-6-isopropoxy-isoquinoline-3-carboxylic acid (2-amino-ethyl)-amide (trifluoro-acetic acid salt); 1-chloro-4-hydroxy-6-isopropoxy-isoquinoline-3-carboxylic acid (2-dimethylamino-ethyl)-amide; 1-chloro-4-hydroxy-7-isopropoxy-isoquinoline-3-carboxylic acid (2-amino-ethyl)-amide (trifluoro-acetic acid salt); 1-chloro-4-hydroxy-7-isopropoxy-isoquinoline-3-carboxylic acid (3-methoxy-ethyl)-amide; 1-chloro-4-hydroxy-7-isopropoxy-isoquinoline-3-carboxylic acid (2-dimethylamino-ethyl)-amide; 1-chloro-4-hydroxy-isoquinoline-7-isopropoxy-isoquinoline-3-carboxylic acid (2-hydroxy-ethyl)-amide; (R)-2-[(1-chloro-4-hydroxy-isoquinoline-3-carbonyl)-amino]-3-hydroxy-propionic acid, (S)-2-[(1-chloro-4-hydroxy-isoquinoline-3-carbonyl)-amino]-3-hydroxy-propionic acid, (R)-2-[(1-chloro-4-hydroxy-6-isopropoxy-isoquinoline-3-carbonyl)-amino]-3-hydroxy-propionic acid, (S)-2-[(1-chloro-4-hydroxy-6-isopropoxy-isoquinoline-3-carbonyl)-amino]-3-hydroxy-propionic acid, (R)-2-[(1-chloro-4-hydroxy-7-isopropoxy-isoquinoline-3-carbonyl)-amino]-3-hydroxy-propionic acid, (S)-2-[(1-chloro-4-hydroxy-7-isopropoxy-isoquinoline-3-carbonyl)-amino]-3-hydroxy-propionic acid, 2-[(1-chloro-4-hydroxy-isoquinoline-3-carbonyl)-amino]-2-methyl-propionic acid, 2-[(1-chloro-4-hydroxy-6-isopropoxy-isoquinoline-3-carbonyl)-amino]-2-methyl-propionic acid, (R)-2-[(1-chloro-4-hydroxy-isoquinoline-3-carbonyl)-amino]-3-(1h-imidazol-4-yl)-propionic acid (trifluoro-acetic acid salt), (S)-2-[(1-chloro-4-hydroxy-isoquinoline-3-carbonyl)-amino]-3-(1h-imidazol-4-yl)-propionic acid (trifluoro-acetic acid salt), (R)-2-[(1-chloro-4-hydroxy-isoquinoline-3-carbonyl)-amino]-3-methyl-butyric acid, (S)-2-[(1-chloro-4-hydroxy-isoquinoline-3-carbonyl)-amino]-3-methyl-butyric acid, (R)-2-[(1-chloro-4-hydroxy-6-isopropoxy-isoquinoline-3-carbonyl)-amino]-3-methyl-butyric acid, (S)-2-[(1-chloro-4-hydroxy-6-isopropoxy-isoquinoline-3-carbonyl)-amino]-3-methyl-butyric acid, (R)-2-[(1-chloro-4-hydroxy-7-isopropoxy-isoquinoline-3-carbonyl)-amino]-3-methyl-butyric acid, (S)-2-[(1-chloro-4-hydroxy-7-isopropoxy-isoquinoline-3-carbonyl)-amino]-3-methyl-butyric acid, (S)-2-[(6-benzyloxy-1-chloro-4-hydroxy-isoquinoline-3-carbonyl)-amino]-3-methyl-butyric acid, (R)-2-[(1-chloro-4-hydroxy-isoquinoline-3-carbonyl)-amino]-3-phenyl-propionic acid, (S)-2-[(1-chloro-4-hydroxy-isoquinoline-3-carbonyl)-amino]-3-phenyl-propionic acid, (R)-2[(1-chloro-4-hydroxy-6-isopropoxy-isoquinoline-3-carbonyl)-amino]-3-phenyl-propionic acid, (S)-2[(1-chloro-4-hydroxy-6-isopropoxy-isoquinoline-3-carbonyl)-amino]-3-phenyl-propionic acid, (R)-2[(1-chloro-4-hydroxy-7-isopropoxy-isoquinoline-3-carbonyl)-amino]-3-phenyl-propionic acid, (S)-2[(1-chloro-4-hydroxy-7-isopropoxy-isoquinoline-3-carbonyl)-amino]-3-phenyl-propionic acid, (R)-2[(1-chloro-4-hydroxy-isoquinoline-3-carbonyl)-amino]-3-(4-hydroxy-phenyl)-propionic acid, (S)-2[(1-chloro-4-hydroxy-isoquinoline-3-carbonyl)-amino]-3-(4-hydroxy-phenyl)-propionic acid, (R)-2[(1-chloro-4-hydroxy-6-isopropoxy-isoquinoline-3-carbonyl)-amino]-3-(4-hydroxy-phenyl-propionic acid, (S)-2[(1-chloro-4-hydroxy-6-isopropoxy-isoquinoline-3-carbonyl)-amino]-3-(4-hydroxy-phenyl-propionic acid, (R)-2[(1-chloro-4-hydroxy-7-isopropoxy-isoquinoline-3-carbonyl)-amino]-3-(4-hydroxy-phenyl-propionic acid, (S)-2[(1-chloro-4-hydroxy-7-isopropoxy-isoquinoline-3-carbonyl)-amino]-3-(4-hydroxy-phenyl)-propionic acid, (R)-2[(1-chloro-4-hydroxy-6-isopropoxy-isoquinoline-3-carbonyl)-amino]-pentanoic acid, (S)-2[(1-chloro-4-hydroxy-6-isopropoxy-isoquinoline-3-carbonyl)-amino]-pentanoic acid, (R)-1-(1-chloro-4-hydroxy-isoquinoline-3-carbonyl)-pyrrolidine-2-carboxylic acid, (S)-1-(1-chloro-4-hydroxy-isoquinoline-3-carbonyl)-pyrrolidine-2-carboxylic acid, (R)-1-(1-chloro-4-hydroxy-6-isopropoxy-isoquinoline-3-carbonyl)-pyrrolidine-2-carboxylic acid, (S)-1-(1-chloro-4-hydroxy-6-isopropoxy-isoquinoline-3-carbonyl)-pyrrolidine-2-carboxylic acid, (R)-6-amino-2-[(1-chloro-4-hydroxy-isoquinoline-3-carbonyl)-amino]-hexanoic acid (trifluoro-acetic acid salt), (S)-6-amino-2-[(1-chloro-4-hydroxy-isoquinoline-3-carbonyl)-amino]-hexanoic acid (trifluoro-acetic acid salt), (R)-6-amino-2[(1-chloro-4-hydroxy-6-isopropoxy-isoquinoline-3-carbonyl)-amino]-hexanoic acid, trifluoroacetic acid salt, (S)-6-amino-2-[(1-chloro-4-hydroxy-6-isopropoxy-isoquinoline-3-carbonyl)-amino]-hexanoic acid (trifluoro-acetic acid, trifluoroacetic acid salt, (S)-6-amino-2-[(1-chloro-4-hydroxy-7-isopropoxy-isoquinoline-3-carbonyl)-amino]-hexanoic acid (trifluoro-acetic acid salt), (R)-2-[(1-chloro-4-hydroxy-isoquinoline-3-carbonyl)-amino]-succinic acid, (S)-2-[(1-chloro-4-hydroxy-isoquinoline-3-carbonyl)-amino]-succinic acid, (R)-2-[(1-chloro-4-hydroxy-6-isopropoxy-isoquinoline-3-carbonyl)-amino]-succinic acid, (S)-2-[(1-chloro-4-hydroxy-6-isopropoxy-isoquinoline-3-carbonyl)-amino]-succinic acid, (R)-2[(6-benzyloxy-1-chloro-4-hydroxy-isoquinoline-3-isoquinoline-3-carbonyl)-amino]-succinic acid, (R)-2-[(6-benzyloxy-1-chloro-4-hydroxy-isoquinoline-3-carbonyl)-amino]-propionic acid, (S)-2-[(7-benzyloxy-1-chloro-4-hydroxy-isoquinoline-3-carbonyl)-amino]-propionic acid, (R)-2-[(7-benzyloxy-1-chloro-4-hydroxy-isoquinoline-3-carbonyl)-amino]-propionic acid, (S)-2-[(1-chloro-4-hydroxy-isoquinoline-3-carbonyl)-amino]-propionic acid, (R)-2-8 (1-chloro-4-hydroxy-isoquinoline-3-carbonyl)-amino]-propionic acid, (S)-2-[(6-isopropoxy-1-chloro-4-hydroxy-isoquinoline-3-carbonyl)-amino]-propionic acid, (R)-2-[6-isopropoxy-1-chloro-4-hydroxy-isoquinoline-3-carbonyl)-amino]-propionic acid, (S)-2-[7-isopropoxy-1-chloro-4-hydroxy-isoquinoline-3-carbonyl)-amino-propionic acid, (R)-2-[(7-isopropoxy-1-chloro-4-hydroxy-isoquinoline-3-carbonyl)-amino]-propionic acid, {[7-(3,5-difluoro-phenoxy)-4-hydroxy-isoquinoline-3-carbonyl]-amino}-acetic acid, {[6-(3,5-difluoro-phenoxy)-4-hydroxy-isoquinoline-3-carbonyl]-amino}-acetic acid, ({7-[4-(4-fluoro-phenoxy)-phenoxy]-4-hydroxy-isoquinoline-3-carbonyl}-amino)-acetic acid, ({6-[4-fluoro-phenoxy)-phenoxy]-4-hydroxy-isoquinoline-3-carbonyl}-amino)-acetic acid, {[7-(3-chloro-4-fluoro-phenoxy)-4-hydroxy-isoquinoline-3-carbonyl]-amino}-acetic acid, {[6-(3-chloro-4-fluoro-phenoxy)-4-hydroxy-isoquinoline-3-carbonyl]-amino}-acetic acid, (S)-2-{[7-(3-fluoro-5-methoxy-phenoxy-)4-hydroxy-isoquinoline-3-carbonyl]-amino}-propionic acid, 2-(S)-[(7-cyclohexyloxy-4-hydroxy-isoquinoline-3-carbonyl)-amino]-propionic acid, 2-(S)-[{(7-(4-fluoro-phenoxy-4-hydroxy-isoquinoline-3-carbonyl]-amino}-propionic acid, 2-(S)-[(4-hydroxy-1-methyl-7-phenoxy-isoquinoline-3-carbonyl)-amino]-propionic acid, 2-(S)-[(4-hydroxy-1methyl-7-phenylsulfanyl-isoquinoline-3-carbonyl)-amino]-propionic acid, 2-(S)-{[(4-hydroxy-7-(4-trifluoromethyl-phenoxy)-isoquinoline-3-carbonyl)-amino}-propionic acid, {[7-(4-chloro-phenoxy-4-hydroxy-1-methyl-isoquinoline-3-carbonyl]-amino}-acetic acid, {[6-(chloro-phenoxy-4-hydroxy-1-methyl-isoquinoline-3-carbonyl]-amino}-acetic acid,{[7-(3,5-difluoro-phenoxy-4-hydroxy-1-methyl-isoquinoline-3-carbonyl]-amino}-acetic acid, {[4-hydroxy-7-(4-methoxy-phenoxy)-1-methyl-isoquinoline-3-carbonyl]-amino}-acetic acid, {[4-hydroxy-6-(4-methoxy-phenoxy)-1-methyl-isoquinoline-3-carbonyl]-amino}-acetic acid, [(6-cyclohexyloxy-4-hydroxy-isoquinoline-3-carbonyl)-amino]-acetic acid, [(7-cyclohexyloxy-4-hydroxy-isoquinoline-3-carbonyl)-amino]-acetic acid, [(7-cyclohexyloxy-4-hydroxy-1-methyl-isoquinoline-3-carbonyl)-amino]-acetic acid, [(7-cyclohexylsulfanyl-4-hydroxy-isoquinoline-3-carbonyl)-amino]-acetic acid, [(7-cyclohexanesulfonyl-4-hydroxy-isoquinoline-3-carbonyl)-amino]-acetic acid, [(4-hydroxy-1-isobutyl-isoquinoline-3-carbonyl)-amino]-acetic acid, [(1-ethyl-4-hydroxy-7-phenoxy-isoquinoline-3-carbonyl)-amino]-acetic acid, [(4-hydroxy-1-methyl-7-phenylsulfanyl-isoquinoline-3-carbonyl)-amino]-acetic acid, {[4-hydroxy-1-methyl-7-(4-trifluoromethyl-phenoxy)-isoquinoline-3-carbonyl]-amino}-acetic acid.

In certain aspects, compounds of the present invention include 4-oxo-[1,10]-phenanthrolines. Exemplary 4-oxo-[1,10]-phenanthrolines are disclosed in, e.g., International Publication NO. WO 03/049686 and International Publication No. WO 03/053997, and include compounds of Formula II

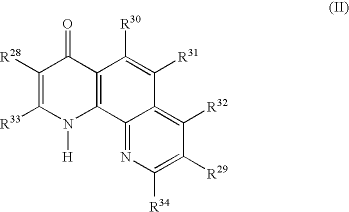

where
R²⁸ is hydrogen, nitro, amino, cyano, halogen, (C₁-C₄)-alkyl, carboxy or a metabolically labile ester derivative thereof; (C₁-C₄)-alkylamino, di-(C₁-C₄)-alkylamino, (C₁-C₆)-alkoxycarbonyl, (C₂-C₄)-alkanoyl, hydroxy-(C₁-C₄)-alkyl, carbamoyl, N-(C₁-C₄)-alkylcarbamoyl, (C₁-C₄)-alkylthio, (C₁-C₄)-alkylsulfinyl, (C₁-C₄)-alkylsulfonyl, phenylthio, phenylsulfinyl, phenylsulfonyl, said phenyl or phenyl groups being optionally substituted with 1to 4identical or different halogen, (C₁-C₄)-alkyoxy, (C₁-C₄)-alkyl, cyano, hydroxy, trifluoromethyl, fluoro-(C₁-C₄)-alkylthio, fluoro-(C₁-C₄)-alkylsulfinyl, fluoro-(C₁-C₄)-alkylsulfonyl, (C₁-C₄)-alkoxy-(C₂-C₄)-alkoxycarbonyl, N,N-di-[(C₁-C₄)-alkyl]-carbamoyl-(C₁-C₄)-alkoxycarbonyl, (C₁-C₄)-alkylamino-(C₂-C₄)-alkoxycarbonyl, di-(C₁-C₄)-alkylamino-(C₂-C₄)-alkoxycarbonyl, (C₁-C₄)-alkoxy-(C₂-C₄)-alkoxy-(C₂-C₄)-alkoxycarbonyl, (C₂-C₄)-alkanoyloxy-C₁-C₄)-alkyl, or n[amino-(C₂-C₄)-alkyl]-carbamoyl;
R²⁹ is hydrogen, hydroxy, amino, cyano, halogen, (C₁-C₄)-alkyl, carboxy or metabolically labile ester derivative thereof, (C₁-C₄)-alkylamino, di-(C₁-C₄)-alkylamino, (C₁-C₆)-alkoxycarbonyl, (C₂-C₄-alkanoyl, (C₁-C₄)-alkoxy, carboxy-(C₁-C₄)-alkoxy, (C₁-C₄)-alkoxycarbonyl-(C₁-C₄)-alkoxy, carbamoyl, N-(C₁-C₈)-alkylcarbamoyl, N,N-di-(C₁-C₈)-alkylcarbamoyl, N-]amino-(C₂-C₈)-alkyl]-carbamoyl, N-[(C₁-C₄)-alkylamino-(C₁-C₈)-alkyl[-carbamoyl, N-[di-(C₁-C₄)-alkylamino-(C₁-C₈)-alkyl]-carbamoyl, N-[cyclopentyl]-carbamoyl, N-(C₁-C₄)-alkylcyclohexylcarbamoyl, N-(C₁-C₄)-alkylcyclopentylcarbamoyl, N-phenylcarbamoyl, N-(C₁-C₄)-alkyl-N-phenylcarbamoyl, N,N-diphenylcarbamoyl, N-]phenyl-(C₁-C₄)-alkyl]-carbamoyl, N-(C₁-C₄)-alkyl-N-[phenyl-(C₁-C₄)-alkyl]-carbamoyl, or N,N-di-[phenyl-(C₁-C₄)-alkyl]-carbamoyl, said phenyl or phenyl groups being optionally substituted with 1to 4identical or different halogen, (C₁-C₄)-alkyoxy, (C₁-C₄)-alkyl, cyano, hydroxy, trifluoromethyl, N-[(C₂-C₄)-alkanoyl]-carbamoyl, N-[(C₁-C₄)-alkoxycarbonyl]-carbamoyl, N-[fluoro-(C₂-C₆)-alkyl]-carbamoyl, N,N-[fluoro-(C₂-C₆)alkyl]-N-(C₁-C₄)-alkylcarbamoyl, N,N-[di-fluoro-(C₂-C₆)-alkyl]carbamoyl, pyrrolidin-1-ylcarbonyl, piperidinocarbonyl, piperazin-1-ylcarbonyl, morpholinocarbonyl, wherein the heterocyclic group is optionally substituted with 1to 4, (C₁-C₄)-alkyl, benzyl, 1,2,3,4-tetrahydro-isoquinolin-2-ylcarbonyl, N,N-[di-(C₁-C₄)-alkyl]-thiocarbamoyl, N-(C₂-C₄)-alkanoylamino, or N-[(C₁-C₄)-alkoxycarbonyl]-amino;
R³⁰ is hydrogen, (C₁-C₄)-alkyl, (C₂-C₄)-alkoxy, halo, nitro, hydroxy, fluoro-(1-4C)alkyl, or pyridinyl;
R³¹ is hydrogen, (C₁-C₄)-alkyl, (C₂-C₄)-alkoxy, halo, nitro, hydroxy, fluoro-(C₁-C₄)-alkyl, pyridinyl, or methoxy; R³² is hydrogen, hydroxy, amino, (C₁-C₄)-alkylamino, di-(C₁-C₄)-alkylamino, halo, (C₁-C₄)-alkoxy-(C₂-C₄)-alkoxy, fluoro-(C₁-C₆)-alkoxy, pyrrolidin-1-yl, piperidino, piperazin-1-yl, or morpholino, wherein the heterocyclic group is optionally substituted with 1to 4identical or different (C₁-C₄)-alkyl or benzyl; and
R³³ and R³⁴ are individually selected from hydrogen, (C₁-C₄)-alkyl, and (C₁-C₄)-alkoxy; including pharmaceutically-acceptable salts, esters, and pro-drugs derived therefrom.

Exemplary compounds of Formula II are described in U.S. Pat. Nos. 5,916,898 and 6,200,974, and International Publication NO. WO 99/21860. All compounds listed in the foregoing patents and publication, in particular, those listed in the compound claims and the final products of the working examples, are hereby incorporated into the present application by reference herein. Exemplary compounds of Formula II include 4-oxo-1,4-dihydro-[1,10]-phenanthroline-3-carboxylic acid (See, e.g., Seki et al. (1974) Chem Abstracts 81:424, No. 21), 3-carboxy-5-hydroxy-4oxo-3,4-dihydro, 1,10-phenanthroline, 3-carboxy-5-methoxy-4-oxo-3,4-dihydro-1,10-phenanthroline, 5-methoxy-4-oxo-1,4-dihydro-[1,10]phenanthroline-3-carboxylic acid ethyl ester, 5-methoxy-4-oxo-1,4-dihydro-[1,10]phenanthroline-3-carboxylic acid, and 3-carboxy-8-hydroxy-4-oxo, 3,4-dihydro-1,10-phenanthroline.

In certain aspects, compounds of the present invention include aryl-sulfono-amino-hydroxamates. Exemplary aryl-sulfono-amino-hydroxamates are disclosed in, e.g., International Publication No. WO 03/049686, International Publication No. WO 03/053997, and International Publication No. WO 04/108121. Such compounds include compounds of Formula III

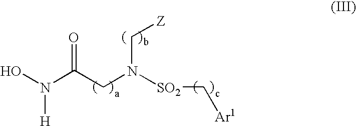

or pharmaceutically acceptable salts thereof, wherein:
a is an integer from 1 to 4;
b is an integer from 0 to 4;
c is an integer from 0 to 4;
Z is selected from the group consisting of (C₃-C₁₀) cycloalkyl, (C₃-C₁₀) cycloalkyl independently substituted with one or more Y¹, 3-10 membered heterocycloalkyl and 3-10 membered heterocycloalkyl independently substituted with one or more Y¹; (C₅-C₂₀) aryl, (C₅-C₂₀) aryl independently substituted with one or more Y¹, 5-20 membered heteroaryl and 5-20 membered heteroaryl independently substituted with one or more Y¹;
Ar¹ is selected from the group (C₅-C₂₀) aryl, (C₅-C₂₀) aryl independently substituted with one or more Y², 5-20 membered heteroaryl and 5-20 membered heteroaryl independently substituted with one or more Y²;
each Y¹ is independently selected from the group consisting of a lipophilic functional group, (C₅-C₂₀) aryl, (C₆-C₂₆) alkaryl, 5-20 membered heteroaryl and 6-26 membered alk-heteroaryl;
each Y² is independently selected from the group consisting of —R′, —OR′, —OR″, —SR′, —SR″, —NR′R′, —NO₂, —CN, -halogen, -trihalomethyl, trihalomethoxy, —C(O)R′, —C(O)OR′, —C(O)NR′R′, —C(O)NR′OR′, —C(NR′R′)═NOR′, —NR′—C(O)R′, —S(O)R°, —SO₂R″, —NR′—SO₂R′, —NR′—C(O)—NR′R′, tetrazol-5-yl, —NR′—C(O)—OR′, —C(NR′R′)═NR′, —S(O)—R′, —S(O)—R″, and —NR′—C(S)—NR′R′; and
each R′ is independently selected from the group consisting of —H, (c₁-C₈) alkyl, (C₂-C₈) alkenyl, and (C₂-C₈) alkynyl; and
each R″ is independently selected from the group consisting of (C₅-C₂₀) aryl and (C₅-C₂₀) aryl independently substituted with one or more —OR′, —SR′, —NR′R′, —NO₂, —CN, halogen or trihalomethyl groups,
or wherein c is 0 and Ar¹ is an N′ substituted urea-aryl, the compound has the structural Formula IIIa;

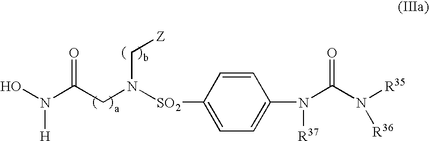

or pharmaceutically acceptable salts thereof, wherein:
a, b, and Z are as defined above; and
R³⁵ and R³⁶ are each independently selected from the group consisting of hydrogen, (C₁-C₈) alkyl, (C₂-C₈) alkenyl, (C₂-C₈) alkynyl, (C₃-C₁₀) cycloalkyl, (C₅-C₂₀) aryl, (C₅-C₂₀) substituted aryl, (C₆-C₂₆) alkaryl, (C₆-C₂₆) substituted alkaryl, 5-20 membered heteroaryl, 5-20 membered substituted heteroaryl, 6-26 membered alk-heteroaryl, and 6-26 membered substituted alk-heteroaryl; and
R³⁷ is independently selected from the group consisting of hydrogen, (C₁-C₈) alkyl, (C₂-C₈) alkenyl, and (C₂-C₈) alkynyl.

Exemplary compounds of Formula III are described in International Publication WO 00/50390. All compounds listed in WO 00/50390, in particular, those listed in the compound claims and the final products of the working examples, are hereby incorporated into the present application by references herein.

Exemplary compounds of Formula II include 3-{[4-(3,4-dibenzyl-ureido)-benzenesulfonyl]-[2-(4-methoxy-phenyl)-ethyl]-amino}-N-hydroxy-propionamide, 3-{[4-(3-chloro-phenyl)-ureido)-benzenesulfonyl]-2-(4-methoxy-phenyl)-ethyl]-amino}-N-hydroxy-propionamide, and 3-{[4-(1,2-diphenyl-ethyl)-ureido]-phenyl)-benzenesulfonyl}-2-(4-methoxy-phenyl)-ethyl]-amino}-N-hydroxy-propionamide.

In certain embodiments, a 2-oxoglutarate mimetic of the present invention is selected from a compound of the Formula IV

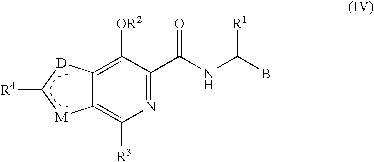

wherein
R¹ are selected from the group consisting of hydrogen (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂—G carboxyl radical, where G is a radical of an alcohol G—OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
R² is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}F_{g}, aryl, heteroaryl, and (C₇-C₁₁)-aralkyl;
one of D or M is —S—, and the other is ═C(R⁵)—;
R³, R⁴, and R⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-arloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂CHFCl, (C₁-C₂₀)-alkylcarbonyl (C₃-C₈)-cycloalkylcarbonyl, (C₆-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenycarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-(C₃-C₈)-cycloalkylcarbamoyl, N-((C₃-C₈)-cycloalkyl-(C₁-C₆)-alkyl)-carbamoyl, N-(+)-dehydroabietylcarbamoyl, N-(C₁-C₆)-alkyl-N-(+)-dehydroabietylcarbamoyl, N-(C₆-C₁₂)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-(C₇-C₁₆)-aralkylcarbamoyloxy, N-((C₁-C₁₀)-alkyl-carbamoyloxy, N-(C₁-C₁₀)-alkyl-N-((C₇-C₁₆)-aralkyloxy-(C₁-C₁₀)-cycloalkylamino, (C₃-C₁₂)-alkenylamino, (C₃-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkyl-aralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N-(C₁-C₁₀)-alkylamino, (C₃-C₈)-cycloalkanoyl-N-(C₁-C₁₀)-alkylamino, (C₆-C₁₂)-N-(C₁-(C₁₀)-alkylamino, (C₇-C₁₁)-aralkanoyl-N-(C₁-C₁₀)-alkylamino, amino(C₁-C₁₀)-alkyl, (C₁-C₂₀)-alkylmercapto, (C₁-C₂₀)-alkylsulfinyl (C₁-C₂₀)-alkylsulfonyl, (C₆-C₁₂)-arylmercapto, (C₆-(C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N-(C₁-C₁₀)-alkylsulfamoyl, N,N-di-(C₁-C₁₀)-alkylsulfamoyl, (C₃-C₈)-cycloalkylsulfamoyl, N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, (C₇-C₁₆)-aralkylsulfonamido, and N-((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[ₓC_{f}H_{(2f+1-g)}F_{g}, —OCF₂Cl, and —OCR₂—CHFCl;
x is 0 to 3;
f is 1to 8;and
g is 0 or 1 to (2f+1);
including the physiologically active salts, esters, and prodrugs derived therefrom.

Compounds of Formula IV include, but are not limited to, [(2-bromo-4-hydroxy-thieno[2,3-c]pyridine-5-carbonyl)-amino]-acetic acid, [(2-bromo-7-hydroxy-thieno[3,2c]pyridine-6-carbonyl)-amino]-acetic acid, {[4-hydroxy-2-(4-methoxy-phenyl)-thieno(2,3-c]pyridine-5-carbonyl]-amino}-acetic acid, {[7-hydroxy-2-(4-methoxy-phenyl)-thieno[2,3-c]pyridine-6-carbonyl]-amino}-acetic acid, [(4-hydroxy-2,7-dimethyl-thieno[2,3-c]pyridine-5-carbonyl)-amino]-acetic acid, [(7-hydroxy-2,4-dimethyl-thieno[3,2-c]-pyridine-6-carbonyl)-amino]-acetic acid, {[7-hydroxy-4-methyl-2-(4-phenoxy-phenyl)-thieno[3,2-c]pyridine-6-carbonyl]-amino}-acetic acid, {[4-hydroxy-2-(4-phenoxy-phenyl)-7-methyl-thieno-[2,3-c]pyridine-5-carbonyl]-amino}-acetic acid, {[4-hydroxy-2-(4-phenoxy-phenyl)-thieno[2,3-c]pyridine-5-carbonyl]-amino}-acetic acid, {[7-hydroxy-2-(4-phenoxy-phenyl)-thieno[3,2-c]pyridine-6-carbonyl]-amino}-acetic acid, [(2,7-dibromo-4-hydroxy-thieno[2,3-c]pyridine-5-carbonyl)-amino]-acetic acid, [(2-bromo-7-chloro-4-hydroxy-thieno[2,3-c]pyridine-5-carbonyl)-amino]-acetic acid, [(7-hydroxy-thieno-[3,2-c]pyridine-6-carbonyl)-amino]-acetic acid, [4-hydroxy-thieno[2,3-c]pyridine-5-carbonyl)-amino]-acetic acid, [(2-bromo-4-chloro-7-hydroxy-thieno[3,2-c]pyridine-6-carbonyl)-amino]-acetic acid, [(2,4-dibromo-7-hydroxy-thieno[3,2-c]pyridine-6-carbonyl)-amino]-acetic acid, [(7-hydroxy-2-phenylsulfanyl-thieno[3,2-c]pyridine-6-carbonyl)-amino]-acetic acid, [(4-hydroxy-2-phenylsulfanyl-thieno[2,3-c]pyridine-5-carbonyl)-amino]-acetic acid, [(4-hydroxy-2,7-diphenyl-thieno[2,3-c]pyridine-5-carbonyl)-amino]-acetic acid, [(7-hydroxy-2,4-diphenyl-thieno[3,2-c]pyridine-6-carbonyl)-amino]-acetic acid, [(7-hydroxy-2-styryl-thieno[3,2-c]pyridine-6-carbonyl)-amino]-acetic acid, [(7-hydroxy-2-phenoxy-thieno-thieno[3,2-c]pyridine-6-carbonyl)amino]-acetic acid, [(7-hydroxy-2-phenethyl-thieno[3,2-c]pyridine-6-carbonyl)-amino]-acetic acid, {[7-hydroxy-2-(3-trifluoromethyl-phenyl)-thieno[3,2-c]pyridine-6-carbonyl]-amino}-acetic acid, {[4-bromo-7-hydroxy-2-(3-trifluoromethyl-phenyl)-thieno[3,2-c]pyridine-6-carbonyl]-amino}-acetic acid, {[4-cyano-7-hydroxy-2-(3-trifluoromethyl-phenyl)-thieno[3,2-c]pyridine-6-carbonyl]-amino}-acetic acid, [(2-cyano-7-hydroxy-thieno[3,2-c]pyridine-6-carbonyl)-amino]-acetic acid, {[7-hydroxy-2-(4-trifluoromethyl-phenyl)-thieno[3,2-c]pyridine-6-carbonyl]-amino}-acetic acid, {[7-hydroxy-2-(2-trifluoromethyl-phenyl)-thieno[3,2-c]pyridine-6-carbonyl]-amino}-acetic acid, {[3(4-fluoro-phenyl)-7-hydroxy-thieno[3,2-c]pyridine-6-carbonyl]-amino}-acetic acid, {[3-(4-fluoro-phenyl)-7-hydroxy-thieno[3,2-c]pyridine-6-carbonyl]-amino}-acetic acid, {[3-(4-fluoro-phenyl)-7-hydroxy-4-methyl-thieno[3,2-c]pyridine-6-carbonyl]-amino}-acetic acid, {[4-cyano-3-(4-fluoro-phenyl)-7-hydroxy-thieno[3,2-c]pyridine-6-carbonyl]-amino)}-acetic acid, {[2-(4-fluoro-phenyl)-7-hydroxy-thieno[3,2-c]pyridine-6-carbonyl]-amino}-acetic acid, {[2-(4-fluoro-phenyl)-7-hydroxy-4-methyl-thieno[3,2-c]-pyridine-6-carbonyl]-amino}-acetic acid, {[2,3-bis-(4-fluoro-phenyl)-7-hydroxy-thieno[3,2-c]pyridine-6-carbonyl]-amino}-acetic acid, {[7-bromo-3-(4-fluoro-phenyl)-4-hydroxy-thieno[2,3-c]pyridine-5-carbonyl]-amino}-acetic acid, {[3-(4-fluoro-phenyl)-4-hydroxy-thieno[2,3-c]pyridine-5-carbonyl]-amino}-acetic acid, {[2-(4-fluoro-phenyl)-4-hydroxy-thieno[2,3]-carbonyl]-amino}-acetic acid, {[2-(4-fluoro-phenyl)-4-hydroxy-7-methyl-thieno[2,3]-pyridine-5-carbonyl]-amino}-acetic acid, [(7-chloro-4-hydroxy-thieno[2,3-c]pyridine-5-carbonyl)-amino]-acetic acid, [(4-chloro-7-hydroxy-thieno[3,2-c]pyridine-6-carbonyl)-amino]-acetic acid, [(7-bromo-4-hydroxy-thieno[2,3-c]pyridine-5-carbonyl)-amino]-acetic acid, [(4-bromo-7-hydroxy-thieno[32,-c]pyridine-6-carbonyl)-amino]-acetic acid, [(4-hydroxy-7-phenyl-thieno[2,3-c]pyridine-5-carbonyl)-amino]-acetic acid, [(7-hydroxy-4-phenyl-thieno[3,2-c]pyridine-6-carbonyl-amino]-acetic acid, {[(7-(4-fluoro-phenyl)-4-hydroxy-thieno[2,3-c]pyridine-5-carbonyl]-amino}-acetic acid, 2-(7-(furan-2-yl-4-hydroxythieno'2,3-c]pyridine-5-carboxamido)acetic acid, carbonyl]-amino}-acetic acid, 2-(7-(furan-2-yl)-4-hydroxythieno[2,3-c]pyridine-5-carboxamido)acetic acid, [(4-furan-2-yl-7-hydroxy-thieno[3,2-c]pyridine-6-carbonyl)-amino]-acetic acid, [(7-furan-3-yl-4-hydroxy-thieno[3,2-c]pyridine-5-carbonyl)-amino]-acetic acid, [(4-furan-3-yl-7-hydroxy-thieno[3,2-c]pyridine-6-carbonyl)-amino]-acetic acid, 2-(4-hydroxy-7-(thiophen-2-yl)thieno[2,3-c]pyridine-5-carboxamido)acetic acid, [(7-hydroxy-4-thiophen-2-yl-thieno[2,3-c]pyridine-5-carbonyl)-amino]-acetic acid, [(4-hydroxy-7-thiophen-3-yl-thieno[2,3-c]pyridine-5-carbonyl)-amino]-acetic acid, [(7-hydroxy-4-thiophen-3-yl-thieno[3,2-c]pyridine-6-carbonyl)-amino]acetic acid, [(4-hydroxy-7-methyl-thieno[2,3-c]pyridine-5carbonyl)-amino]-acetic acid, [(7-hydroxy-4methyl-thieno[3,2-c]pyridine-6carbonyl)-amino]-acetic acid, [(7-ethynyl-4-hydroxy-thieno[2,3-c]pyridine-5-carbonyl)-amino]-acetic acid, [(4-ethynyl-7-hydroxy-thieno[32,-c]pyridine-6-carbonyl)-amino]-acetic acid, [(7-cyano-4-hydroxy-thieno[2,3-c]pyridine-5-carbonyl)-amino]-acetic acid, and [(4-cyano-7-hydroxy-thieno[32,-c]pyridine-6-carbonyl)-amino]-acetic acid.

Exemplary compounds for use in the present methods include Compound A(1-Chloro-4-hydroxy-isoquinoline-3-carbonyl)-amino]acetic acid; Compound B(S)-2-[(4-Hydroxy-7-phenoxy-isoquinoline-3-carbonyl)-amino]-propionic acid; Compound C {[4-Hydroxy-7-(4-methoxy-phenoxy)-isoquinoline-3-carbonyl]-amino}-acetic acid; Compound D [(4-Hydroxy-1-methyl-7-phenoxy-isoquinoline-3-carbonyl)-amino]-acetic acid; and Compound E [7-(4-Fluoro-phenoxy)-4-hydroxy-isoquinoline-3-carbonyl]-amino-acetic acid.

Unless otherwise specified, the term “alkyl” as used herein refers to monovalent alkyl groups having from 1 to 10 carbon atoms, preferably from 1 to 5 carbon atoms and more preferably 1 to 3 carbon atoms. This term is exemplified by groups such as methyl, ethyl, n-propyl, iso-propyl, n-butyl, t-butyl, n-pentyl and the like. The term “substituted alkyl” unless otherwise specified is used herein to refer to an alkyl group, of from 1 to 10 carbon atoms, preferably, 1 to 5carbon atoms, having from 1 to 5 substituents, preferably 1 to 3 substituents, independently selected from the group consisting of alkoxy, substituted alkoxy, acyl, acylamino, acyloxy, amino, substituted amino, aminoacyl, aminocarbonylamino, aminothiocarbonylamino, aminocarbonyloxy, aryl, substituted aryl, aryloxy, substituted aryloxy, aryloxaryl, substituted aryloxaryl, cyano, halogen, hydroxyl, nitro, oxo, thioxo, carboxyl, carboxyl esters, cycloalkyl, substituted cycloalkyl, thiol, alkylthio, substituted alkylthio, arylthio, substituted arylthio, cycloalkylthio, substituted cycloalkythio, heteroarylthio, substituted heterarylthio, heterocyclicthio, substituted heterocyclicthio, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂alkyl, —OS(O)₂-substituted alkyl, —OS(O)₂-aryl, —OS(O)₂-substituted aryl, OS(O)₂-heteroaryl, —OS(O)₂-substituted heteroaryl, —OS(O)₂-heterocyclic, —OS(O)₂-heterocyclic, —OSO₂—NR⁴⁰R⁴⁰ where each R⁴⁰ is hydrogen or alkyl, —NR⁴⁰S(O)₂-alkyl, —NR⁴⁰S(O)₂-substituted alkyl, —NR⁴⁰S(O)₂-aryl, —NR⁴⁰S(O)₂-substituted aryl, —NR⁴⁰S(O)₂-heteroaryl, —NR⁴⁰S(O)₂-substituted heteroaryl —NR⁴⁰S(O)₂-heterocyclic, —NR⁴⁰S(O)₂-substituted heterocyclic, —NR⁴⁰S(O)₂—NR⁴⁰-alkyl, —NR⁴⁰S(O)₂—NR⁴⁰—substituted alkyl, —NR⁴⁰S(O)₂-NR⁴⁰-aryl, —NR⁴⁰S(O)₂-NR⁴⁰-substituted aryl, —NR⁴⁰S(O)₂-NR⁴⁰-heteroaryl, —NR⁴⁰S(O)₂-NR⁴⁰-substituted heteroaryl, —NR⁴⁰S(O)₂-NR⁴⁰-heterocyclic, and —NR⁴⁰S(O)₂-NR⁴⁰-substituted heterocyclic where each R⁴⁰ is hydrogen or alkyl.

“Alkoxy” unless otherwise specified is used herein to refer to the group “alkyl-O—” which includes, by way of example, methoxy, ethoxy, n-propoxy, iso-propoxy, n-butoxy, t-butoxy, sec-butoxy, n-pentoxy and the like.

“Substituted alkoxy” unless otherwise specified is used herein to refer to the group “substituted alkyl—O—”.

“Acyl” unless otherwise specified is used herein to refer to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl—C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)— provided that a nitrogen atom of the heterocyclic or substituted heterocyclic is not bound to the —C(O)— group wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

The terms “aminoacyl” or, as a prefix, “carbamoyl” or “carboxamide,” or “substituted carbamoyl” or “substituted carboxamide,” are used herein unless otherwise specified to refer to the group —C(O)NR¹⁴²R¹⁴² where each R¹⁴² is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R¹⁴² is joined to form together with the nitrogen atom a heterocyclic or substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” unless otherwise specified is used herein to refer to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” unless otherwise specified is used herein to refer to alkenyl group preferably having from 2 to 6 carbon atoms and more preferably 2 to 4 carbon atoms and having at least 1 and preferably from 1 to 2 sites or alkenyl unsaturation. “Substituted alkenyl” unless otherwise specified is used herein to refer to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Alkynyl” unless otherwise specified is used herein to refer to alkynyl group preferably having from 2 to 6 carbon atoms and more preferably 2 to 3 carbon atoms and having at least 1 and preferably from 1-2 sites of alkynyl unsaturation.

“Substituted alkynyl” unless otherwise specified is used herein to refer to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Amino” refers to the group —NR₂.

“Substituted amino” unless otherwise specified is used herein to refer to the group —NR¹⁴¹R¹⁴¹, where each R¹⁴¹ group is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, provided that both R¹⁴¹ groups are not hydrogen; or the R¹⁴¹ groups can be joined together with the nitrogen atom to form a heterocyclic or substituted heterocyclic ring.

“Acylamino” unless otherwise specified is used herein to refer to the group —NR¹⁴⁵C(O)alkyl, —NR¹⁴⁵C(O)substituted alkyl, —NR¹⁴⁵C(O)cycloalkyl, —NR¹⁴⁵C(O)substituted cycloalkyl, —NR¹⁴⁵C(O)alkenyl, —NR¹⁴⁵C(O)substituted alkenyl, —NR¹⁴⁵C(O)alkynyl, —NR¹⁴⁵C(O)substituted alkynyl, —NR¹⁴⁵C(O)aryl, —NR¹⁴⁵C(O)substituted aryl, —NR¹⁴⁵C(O)heteroaryl, —NR¹⁴⁵C(O)substituted heteroaryl, —NR¹⁴⁵C(O)heterocyclic, and —NR¹⁴⁵C(O)substituted heterocyclic where R¹⁴⁵ is hydrogen, or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are defined herein.

“Carbonyloxyamino” unless otherwise specified is used herein to refer to the groups —NR¹⁴⁶C(O)O-alkyl, —NR¹⁴⁶C(O)O-substituted alkyl, —NR¹⁴⁶C(O)O-alkenyl, —NR¹⁴⁶C(O)O-substituted alkenyl, —NR¹⁴⁶C(O)O-alkynyl, —NR¹⁴⁶C(O)O-substituted alkynyl, —NR¹⁴⁶C(O)O-cycloalkyl, —NR¹⁴⁶C(O)O-substituted cycloalkyl, —NR¹⁴⁶C(O)O-aryl, —NR¹⁴⁶C(O)O-substituted aryl, —NR¹⁴⁶C(O)O-heteroaryl, —NR¹⁴⁶C(O)O-substituted heteroaryl, —NR¹⁴⁶C(O)O-heterocyclic, and —NR¹⁴⁶C(O)O-substituted heterocyclic where R¹⁴⁶ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy,” or, as a prefix, “carbamoyloxy,” or “substituted carbamoyloxy,” are used herein unless otherwise specified to refer to the groups —OC(O)NR¹⁴⁷R¹⁴⁷ where R¹⁴⁷ is independently hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic or where each R¹⁴⁷ is joined to form, together with the nitrogen atom a heterocyclic or substituted heterocyclic and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” unless otherwise specified is used herein to refer to the group —NR¹⁴⁹C(O)NR¹⁴⁹—where R¹⁴⁹ is selected from the group consisting of hydrogen and alkyl.

“Aryl” or “Ar” unless otherwise specified are used herein to refer to a monovalent aromatic carbocyclic group of from 6 to 14 carbon atoms having a single ring (e.g., phenyl) or multiple condensed rings (e.g., naphthyl or anthryl), which condensed rings may or may not be aromatic (e.g., 2-benzoxazolinone, 2H-1,4-benzoxazin-3(4H)-one-7yl, and the like), provided that the point of attachment is the aryl group. Preferred aryls include phenyl and naphthyl.

“Substituted aryl” unless otherwise specified is used herein to refer to aryl groups, as defined herein, which are substituted with from 1 to 4, preferably 1-3, substituents selected from the group consisting of hydroxy, acyl, acylamino, carbonylaminothio, acyloxy, alkyl, substituted alkyl, alkoxy, substituted alkoxy, alkenyl, substituted alkenyl, alkynyl, substituted alkynyl, amidino, amino, substituted amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteraryloxy, substituted heteroaryloxy, heterocyclyloxy, substituted heterocyclyloxy, carboxyl, carboxyl esters cyano, thiol, alkylthio, substituted alkylthio, arylthio, substituted arylthio, heteroarylthio, substituted heteroarylthio, cycloalkylthio, substituted cycloalkylthio, heterocyclicthio, substituted heterocyclicthio, cycloalkyl, substituted cycloalkyl, guanidino, halo, nitro, heteroaryl, substituted heteroaryl, heterocyclic, substituted heterocyclic, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂NR¹⁵¹R¹⁵¹ where each R¹⁵¹ is a hydrogen or alkyl, —NR¹⁵¹S(O)₂-alkyl, —NR¹⁵¹S(O)₂-substituted alkyl, —NR¹⁵¹S(O)₂-aryl, —NR¹⁵¹S(O)₂-substituted aryl, —NR¹⁵¹S(O)₂-heteroaryl, —NR¹⁵¹S(O)₂-substituted heteroaryl, —NR¹⁵¹S(O)₂-heterocyclic, —NR¹⁵¹S(O)₂-substituted heterocyclic, —NR¹⁵¹S(O)₂—NR¹⁵¹-alkyl, —NR¹⁵¹S(O)₂—NR¹⁵¹-substituted alkyl, —NR¹⁵¹S(O)₂—NR¹⁵¹-aryl, —NR¹⁵¹S(O)₂—NR¹⁵¹-substituted aryl, —NR¹⁵¹S(O)₂—NR¹⁵¹-heteroaryl, —NR¹⁵¹S(O)₂—NR¹⁵¹-substituted heteroaryl, —NR¹⁵¹S(O)₂—NR¹⁵¹-heterocyclic, —NR¹⁵¹S(O)₂—NR¹⁵¹-substituted heterocyclic where each R¹⁵¹ is hydrogen or alkyl, wherein each of the terms is as defined herein.

“Aryloxy” unless otherwise specified is used herein to refer to the group aryl-O— that includes, by way of example, phenoxy, naphthoxy, and the like.

“Substituted aryloxy” unless otherwise specified is used herein to refer to substituted aryl-O—aryl.

“Aryloxyaryl” unless otherwise specified is used herein to refer to the group -aryl-O-aryl.

“Substituted aryloxyaryl” unless otherwise specified is used herein to refer to aryloxyaryl groups substituted with form 1 to 3 substituents on either or both aryl rings as defined above for substituted aryl.

“Carboxyl” refers to —COOH or salts thereof.

“Carboxyl esters” unless otherwise specified is used herein to refer to the groups —C(O)O-alkyl, —C(O)O-substituted alkyl, —C(O)O-aryl, and —C(O)O-substituted aryl wherein alkyl, substituted alkyl, aryl and substituted aryl are as defined herein.

“Cycloalkyl” unless otherwise specified is used to refer to cyclic alkyl groups of from 3 to 10 carbon atoms having single or multiple cyclic rings including, by way of example, adamantyl, cyclopropyl, cyclobutyl, cyclopentyl, cyclooctyl and the like.

“Substituted cycloaklyl” unless otherwise specified is used herein to refer to a cycloalkyl group, having from 1 to 5 substituents selected from the group consisting of oxo (═O), thioxo (═S), alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Cycloalkoxy” unless otherwise specified is used herein to refer to —O-cycloalkyl groups.

“Substituted cycloalkoxy” unless otherwise specified is used herein to refer to —O-substituted cycloalkyl groups.

“Halo” or “halogen” refer to fluoro, chloro, bromo and iodo and, preferably, fluoro or chloro.

“Heteroaryl” unless otherwise specified is used to refer to an aromatic group of from 1 to 15 carbon atoms, preferably from 1 to 10 carbon atoms, and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl). Preferred heteroaryls include pyridinyl, pyrrolyl, indolyl, thiophenyl, and furyl.

“Substituted heteroaryl” unless otherwise specified is used herein to refer to heteroaryl groups that are substituted with from 1 to 3 substituents selected from the same group of substituents defined for substituted aryl.

“Heteroaryloxy” unless otherwise specified is used herein to refer to the group —O-heteroaryl and “substituted heteroaryloxy” refers to the group —O-substituted heteroaryl.

“Heterocycle” or “heterocyclic” unless otherwise specified are used herein to refer to a saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl provided that the point of attachment is at the heterocycle.

“Substituted heterocyclic” unless otherwise specified is used to refer to heterocycle groups that are substituted with from 1 to 3 of the same substituents as defined for substituted cycloalky.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinolin, quinolin, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” unless otherwise specified is used herein to refer to the group —O-heterocyclic and “substituted heterocyclyloxy” refers to the group —O-substituted heterocyclic.

“Thiol” or “mercapto” refer to the group —SH.

“Alkysulfanyl” and “alkylthio” unless otherwise specified are used herein to refer to the groups —S-alkyl where alkyl is as defined above.

“Substituted alkylthio” and “substituted alkylsulfanyl” unless otherwise specified are used herein to refer to the grop —S-substituted alkyl is as defined above.

“Cycloalkylthio” or “cycloalkylsulfanyl” unless otherwise specified are used herein to refer to the groups —S-cycloalkyl where cycloalkyl is as defined above.

“Substituted cycloalkylthio” unless otherwise specified is used herein to refer to the group —S-substituted cycloalkyl where substituted cycloalkyl is as defined above.

“Arylthio” unless otherwise specified is used herein to refer to the group —S-aryl and “substituted arylthio” unless otherwise specified is used herein to refer to the group —S-substituted aryl where aryl and substituted aryl are as defined above.

“Heteroarylthio” unless otherwise specified is used herein to refer to the group —S-heteroaryl and “substituted heteroarylthio” unless otherwise specified is used herein to refer to the group —S-substituted heteroaryl where heteroaryl and substituted heteroaryl are as defined above.

“Heterocyclicthio” unless otherwise specified is used to refer to the group —S-heterocyclic and “substituted heterocyclicthio” unless otherwise specified is used herein to refer to the group —S-substituted heterocyclic where heterocyclic and substituted heterocyclic are as defined above.

The term “amino acid” refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (ed.), Synthesis of Optically Active .alpha.-Amino Acids, Pergamon Press (1989); Evans et al., J. Amer. Chem. Soc., 112:4011-4030 (1990); Pu et al., J. Amer. Chem. Soc., 56:1280-1283 (1991); Williams et al., J. Amer. Chem. Soc., 113:9276-9286 (1991); and all references cited therein. The present invention includes the side chains of unnatural amino acids as well.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “prodrug” refers to compounds of this invention which have been modified to include a physiologically and biocompatible removable group which group is removed in vivo to provide for the active drug, a pharmaceutically acceptable salt thereof or a biologically active metabolite thereof. Suitable removable groups are well known in the art and particularly preferred removable groups include esters of the carboxylic acid moiety on the glycine substituent. Preferably such esters include those derived from alkyl alcohols, substituted alkyl alcohols, hydroxy substituted aryls and heteroaryls and the like. Another preferred removable group are the amides formed from the carboxylic acid moiety on the glycine substituent. Suitable amides are derived from amines of the formula HNR²⁰R²¹ where R²⁰ and R²¹ are independently hydrogen, alkyl, substituted alkyl, aryl, substituted aryl, and the like.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to—substituted aryl-(substituted aryl)-substituted aryl.

Similarly, it is understood that the above definitions are not intended to include impermissible substitution patterns (e.g., methyl substituted with 5 fluoro groups or a hydroxyl group alpha to ethenylic or acetylenic unsaturation). Such impermissible substitution patterns are well known to the skilled artisan.

### Methods for Identifying Compounds

Methods for identifying compounds of the invention are also provided. Assays for hydroxylase activity are standard to the art. Such assays can directly or indirectly measure hydroxylase activity. For example, an assay can measure hydroxylated residues, e.g., proline, asparagine, etc., present in the enzyme substrate, e.g., a target protein, a synthetic peptide mimetic, or a fragment thereof. (See, e.g., Palmerini et al. (1985) J Chromatogr 339:285-292.) A reduction in hydroxylated residue, e.g., proline or asparagine, in the presence of a compound is indicative of a compound that inhibits hydroxylase activity. Alternatively, assays can measure other products of the hydroxylation reaction, e.g., formation of succinate form 2-oxoglutarate. (See, e.g., Cunliffe et al. (1986) Biochem J 240:617-619.) Kaule and Gunzler (1990); Anal Biochem 184:291-297) describe an exemplary procedure that measures production of succinate from 2-oxoglutarate.

Procedures such as those described above can be used to identify compounds that modulate HIF hydroxylase activity. Target protein may include HISα or a fragment thereof, e.g., HIF(556-575). Enzyme may include, e.g, HIF prolyl hydroxylase (see, e.g., GenBank Accession No. AAG33965, etc.) or HIF asparaginyl hydroxylase (see, e.g., GenBank Accession No. AAL27308, etc.). obtained from any source. Enzyme may also be present in a crude cell lysate or in a partially purified form. For example, procedures that measure HIF hydroxylase activity are described in Ivan et al. (2001, Science 292:464-468; and 2002, Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIFα.

For clarity, an agent to use in the present methods is any compound that stabilizes HIFα. Methods for determining whether or not a particular agent stabilizes HIFαare available in the art and are described, supra.

### Modes of Administration

The compositions of the present invention can be delivered directly or in a pharmaceutical compositions containing excipients, as is well known in the art. The present methods of treatment involve administration of an effective amount of a compound of the present invention to a subject having or at risk for having cancer-related anemia, i.e., anemia of cancer.

An effective amount, e.g., dose, of compound or drug can readily be determined b routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In preferred embodiments, the compounds of the present invention are administered orally. For example, in certain embodiments, the invention provides for oral administration of a compound selected from the group consisting of: Compound A (1-Chloro-4hydroxy-isoquinoline-3-carbonyl)-amino]-acetic acid; Compound B (S)-2-[(4-Hydroxy-7-phenoxy-isoquinoline-3-carbonyl)-amino]-propionic acid; Compound C {[4-Hydroxy-7-(4-methoxy-phenoxy)-isoquinoline-3-carbonyl]-amino}-acetic acid; and Compound E [7-(4-Fluoro-phenoxy)-4hydroxy-isoquinoline-3-carbonyl]-amino-acetic acid.

Pharmaceutical dosage forms of a compound of the invention may be provided in an instant release, controlled release, sustained release, or target drug-delivery system. Commonly used dosage forms include, for example, solutions and suspensions (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefor, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include on or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g, containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl, pyrrolidone, silicates, silicium dioxide, sodium benzoate, sorbitol, starches, stearic acid or a salt thereof, sugars (i.e., dextrose, sucrose, lactos, etc.), talc, traganth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol form pressured packs, or a nebuliser, usually with the use of a propellant, e.g., halogenated carbons dervided from methan and ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identify of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects in the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal, or human that is being sought by the researcher, veterinarian, medical doctor, or other clinician, e.g., an increase in hemoglobin levels, an increase in hematocrit, amelioration of the symptoms of cancer-related anemia, etc.

Dosages preferably fall within a range of circulating concentrations that include the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject 'condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro date and animal experiments. Dosages necessary to achieve the MEC will depend on individual characteristics and route of administration. In cases of local administration or selective uptake, the effective local concentration of the drug may not be related to plasma concentration.

In some embodiments of the present invention, effective doses for preferred compounds of the invention (e.g., Compound A, Compound B, Compound C, Compound D, and Compound E) include 3 mg/kg, 6 mg/kg, 10 mg/kg, 15 mg/kg, 20 mg/kg, and 30 mg/kg. These doses are therefore particularly preferred for use in the present invention.

In additional embodiments, effective treatment regimes for preferred compounds of the invention (e.g., Compound A, Compound B, Compound C, Compound D, and Compound E) include administration two or three times weekly. These regimes are therefore particularly preferred for use in the present invention.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope of the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1

### Compounds and Methods of the Invention Are Effective at Treating Anemia of Cancer

To examine the effectiveness of methods and compounds of the present invention at preventing the development of cancer-related anemiaanemia of cancer or in treating anemia of cancer, the following experiments were performed. Immuno-compromised athymic CD-1 nu/nu nude mice males (5-6 weeks old) were used in this study (Charles River Laboratories, Wilmington, Mass.). Animals were maintained in a HEPA-filtered environment during the experimental period. Cages, food, and bedding were autoclaved. Animal diets were obtained from Harlan Teklad (Madison, Wis.). Hydrochloric acid, 0.15% (v/v), was added to the drinking water.

Compounds of the invention were pre-formulated in an aqueous vehicle consisting of 0.1% (w/w) Polysorbate 80 (J T Baker) and 0.5% (w/w) high viscosity carboxymethyl cellulose sodium (Spectrum) to achieve a final 10 ml/kg dosing (oral gavage).

The human H-460 lung cancer cell line used was obtained from the National Cancer Institute. (Brower et al., (1986) Cancer Res 46:798-806.) A stock tumor was established by subcutaneously injection a cell suspension into nude mice. The resulting tumor was maintained in nude mice subcutaneously as tumor stick prior to use. Tumor implantation was performed when the stock tumors were in log phase of growth. Before implantation, tumor tissue was harvested from stock mice and placed in RPMI-1640 medium. Necrotic tissues were dissected away and viable tissues were cut into 1-2 mm² pieces. Tumor fragments were then transplanted subcutaneously to the right flank of the nude mice.

Treatment (administration of compounds of the present invention) was stated when the inoculated tumors reached approximately 100 mm³, and continued for four weeks. Table 1 below show the study design and treatments used in each group.

TABLE 1

| Agent | Dose | Schedule | Route | n |
|---|---|---|---|---|
| CMC Vehicle | 10 ml/kg | M, W, F × 4 | PO | 10 |
| Cmpd A | 20 mg/kg | M, W, F × 4 | PO | 10 |
| Cmpd A | 60 mg/kg | M, W, F × 4 | PO | 10 |
| Cmpd B | 6 mg/kg | M, W, F × 4 | PO | 10 |
| Cmpd B | 20 mg/kg | M, W, F × 4 | PO | 10 |
| Cmpd C | 20 mg/kg | M, W, F × 4 | PO | 10 |

PO, oral gavage;
i.v., intravenous infusion;
s.c., subcutaneous injection.

Table 2 below shows results of the effect of compound administration on various blood parameters associated with anemia. These include red blood cell counts, hemoglobin, hematocrit, mean corpuscular volume, mean corpuscular hemoglobin, mean corpuscular hemoglobin concentration, red blood cell distribution, reticulocytes, reticulocyte counts, and immature reticulocyte fraction. Table 2 shows that non-treated control animals with cancer had reduced levels of these key measurable blood parameters, indicating the animals had cancer-related anemia (i.e. anemia of cancer). As shown in Table 2, blood parameters determined at the study endpoint were affected by treatment with compounds of the invention as well. Key markers of increased and effective erythropoiesis were consistently increased in animals treated with a compound of the present invention compared to that in control animals. These markers of increased and effective erythropoiesis included increased red blood cell counts (RBC), increased hemoglobin content (HGB), and increased hematocrit (HCT). One treatment group (Compound C, 20 mg/kg) met statistical significance cutoffs for change vs. the vehicle control grop for both RBC and HGB (p<0.05, Dunnett's ANOVA). These results demonstrated that methods and compounds of the present invention are useful for treating anemia of cancer.

TABLE 2

| | | Cmpd | Cmpd | | | |
|---|---|---|---|---|---|---|
| | | A | A | | | |
| | Vehicle | 20 | 60 | Cmpd B | Cmpd B | Cmpd C |
| | control | mg/kg | mg/kg | 6 mg/kg | 20 mg/kg | 20 mg/kg |
| RBC | 8.11 | 8.84 | 8.60 | 8.71 | 8.78 | 9.36 |
| (×10⁶/ul) | | | | | | |
| HGB | 12.1 | 13.3 | 13.5 | 12.7 | 12.7 | 14.0 |
| (g/dl) | | | | | | |
| HCT (%) | 35.5 | 40.4 | 39.5 | 37.7 | 37.7 | 41.1 |
| MCV (fl) | 43.8 | 45.6 | 45.8 | 43.2 | 43.0 | 43.8 |
| MCH (pg) | 15.0 | 15.1 | 15.6 | 14.6 | 14.5 | 14.9 |
| MCHC | 34.3 | 33.1 | 34.2 | 33.8 | 33.7 | 34.1 |
| (g/dl) | | | | | | |
| RDW (%) | 21.3 | 21.3 | 23.6 | 21.4 | 21.7 | 22.3 |
| RET (%) | 9.0 | 6.4 | 6.5 | 7.5 | 6.3 | 4.9 |
| Abs Retic | 707 | 559 | 539 | 653 | 552 | 457 |
| IRF | 0.65 | 0.63 | 0.65 | 0.64 | 0.56 | 0.56 |

Abbreviations:
RBC, red blood cells;
HGB, hemoglobin;
HCT, hematocrit;
MCV, mean corpuscular volume;
MCH, mean corpuscular hemoglobin;
MCHC, MCH concentration;
RDW, RBC distribution width;
RET, reticulocytes;
Abs Retic, reticulocyte count;
IRF, immature RET fraction.

### Example 2

### Compounds and Methods of the Invention Are Effective at Treating Anemia of Cancer

To examine the effectiveness of methods and compounds of the present invention at preventing the development of anemia of cancer or in treating anemia of cancer, the following experiments were performed. Immuno-compromised athymic CD-1 nu/nu nude mice males (5-6 weeks old) were used in this study (Charles River Laboratories, Wilmington, Mass.). Animals were maintained in a HEPA-filtered environment during the experimental period. Cages, food, and bedding were autoclaved. Animal diets were obtained from Harlan Teklad (Madison, Wis.). Hydrochloric acid, 0.15% (v/v), was added to the drinking water.

Compounds of the invention were formulated in an aqueous vehicle consisting of 0.1% (w/w) Polysorbate 80 (J T Baker) and 0.5% (w/w) high viscosity carboxymethyl cellulose sodium (Spectrum) to achieve a final 10 ml/kg dosing (oral gavage).

The A549 human lung cancer cell line was used. This cell line has been characterized extensively. (See, e.g., Kraus-Berthier et al. (2000) Clin Cancer Res 6:297-304; Hanze et al. (2003) Biochem Biophys Res Commun 312:571-577; Wedge et al. (2002) Cancer Res 62::4645-4655; and Abdollahi et al. (2003) Cancer Res 63:8890-8898.)

A stock tumor was developed by subcutaneously injecting an A549 cell suspension into stock mice. The resulting tumor was maintained subcutaneously as tumor stock prior to use. Tumor implantation was performed when the stock tumors were in log phase of growth. Before implantation, tumor tissue was harvested from stock mice and placed in RPMI-1640 medium. Necrotic tissues were dissected away and viable tissues were cut into 1-2 mm² pieces. Tumor fragments were then transplanted subcutaneously to the right flank of the nude mice.

Administration of compounds of the present invention (administered three-times per week) was initiated when the inoculated tumors reached approximately 100 mm³, and continued for four weeks thereafter. Table 3 below shows the study design and treatments used in each group.

Plasma was isolated from the animals 6 hours following administration of the final dose of compound. Plasma erythropoietin (EPO) levels were determined by ELISA. As shown in Table 3, vehicle-treated control mice implanted with A459 human lung tumors had a mean plasma EPO concentration of 242 pg/ml. Animals administered compounds of the present invention (Compound A, 20 mg/kg or 60 mg/kg; Compound C, 20 mg/kg; or Compound D, 20 mg/kg or 60 mg/kg) had increased plasma EPO levels compared to that of vehicle-treated control animals. (See Table 3.)

TABLE 3

| | Mean [EPO] | |
|---|---|---|
| Group | (pg/ml) | SEM |
| Vehicle | 242 | 76 |
| Cmpd A 20 mg/kg | 569 | 180 |
| Cmpd A 60 mg/kg | 5059* | 1600 |
| Cmpd D 6 mg/kg | 455 | 152 |
| Cmpd D 20 mg/kg | 489 | 155 |
| Cmpd C 20 mg/kg | 3581* | 1132 |

Values represent means +/− SEM, standard error of the mean (n = 10)
*= pval < 0.05, ANOVA vs. vehicle (Dunnett's).

These results showed that administration of compounds of the present invention to animals subcutaneously-implanted with human A549 lung tumors resulted in a dose-dependent increase in circulating EPO levels. Taken together, these results demonstrated that methods and compounds of the present invention effectively increased endogenous EPO levels in animals with cancer. Additionally, these results suggested that the present methods and compounds are useful for effective treatment of anemia of cancer by increasing endogenous EPO.

### Example 3

### Compounds and Methods of the Invention Are Effective at Treating Anemia of Cancer

The effectiveness of administration of compounds of the present invention at preventing the development of anemia of cancer or at treating anemia of cancer in an animal model of cancer was examined. This experiment was carried out as described above in Example 1, with the following modifications. Genetically-engineered tumors of GFP-transfected H-460 cells were used in this study. The human H-460 lung cancer cell line used was obtained from the National Cancer Institute. (Brower et al., (1986) Cancer Res 46:798-806.) 1-2 mm³ H-460-GFP lung tumor fragments were implanted by surgical orthotopic implantation (SOI) and sutured directly into lung tissue. (Yang et al., (1998) Cancer Res 58:4217-4221.) Compound dosing was initiated immediately after successful tumor implantation and growth, confirmed by fluorescent GFP imaging (−5 days following tumor implantation). Compounds were administered three-times per week.

Plasma was isolated from these animals 6 hours following administration of the final dose of compound. Plasma erythropoietin (EPO) levels were determined by ELISA. As shown below in Table 4, vehicle-treated control mice implanted with H-460-GFP human lung tumors (orthotopically xenografted) had a mean plasma EPO concentration of 424 pg/mg. Animals administered compounds of the present invention (Compound A, 20 mg/kg or 60 mg/kg; Compound C, 20 mg/kg; Compound D, 20 mg/kg; or Compound E, 20 mg/kg) had elevated plasma EPO levels compared to that of vehicle-treated control animals. (See Table 4.)

TABLE 4

| | Average [EPO] | |
|---|---|---|
| Group | (pg/ml) | std.dev |
| CMC Vehicle (n = 5) | 424 | 298 |
| Cmpd A 20 mg/kg (n = 5) | 1752 | 1143 |
| Cmpd A 60 mg/kg (n = 7) | 3454 | 1201 |
| Cmpd E 20 mg/kg (n = 8) | 18720 | 8839 |
| Cmpd D 20 mg/kg (n = 9) | 2176 | 2838 |
| Cmpd C 20 mg/kg (n = 8) | 5898 | 2327 |
| rhEPO (n = 7) | 215 | 49 |
| CMC Vehicle (non-tumored) (n = 8) | 230 | 87 |

Values represent means +/− SEM.
*= pval < 0.05, ANOVA vs. vehicle (Dunnett's)

These results demonstrated that methods and compounds of the present invention effectively increased endogenous EPO levels in animals with cancer. Additionally, these results suggested that the present methods and compounds are useful for effective treatment of anemia of cancer by increasing enodgenous EPO.

Whole blood was collected from these animals 6 hours following administration of the final dose of compound. Various measurable blood parameters indicative of erythropoiesis and treatment of anemia (red blood cell count, hemoglobin, and hematocrit) were analyzed.

Table 5 below shows the effect of administration of compounds of the present invention on red blood cell count (RBC), hemoglobin (HGB), and hematocrit (HCT) levels in an animal model of cancer of anemia. As shown in Table 5, RBC, HGB, and HCT levels were reduced in vehicle-treated animals with tumors compared to that observed in control (non-tumor) animals. These results indicated that the presence of tumors (i.e., cancer) in a mouse xenograft model of cancer reduced erythropoiesis and resulted in the development of anemia of cancer.

TABLE 5

| Group | RBC | HGB (g/dL) | HCT (%) |
|---|---|---|---|
| Control (n = 10) | 10.3 +/− 1 | 16 +/− 1.2 | 48.7 +/− 4.5 |
| Vehicle (n = 5) | 9.4 +/− 0.4 | 14.9 +/− 0.6 | 43.5# +/− 2.0 |
| Cmpd A 20 mg/kg | 9.7 +/− 0.8 | 15.9 +/− 0.8 | 47.0 +/− 2.6 |
| (n = 7) | | | |
| Cmpd A 60 mg/kg | 10.3 +/− 1.0 | 16.9* +/− 1.3 | 49.0* +/− 4.0 |
| (n = 9) | | | |
| Cmpd E 20 mg/kg | 10.3 +/− 1.0 | 17.4* +/− 0.7 | 50.5* +/− 3.9 |
| (n = 8) | | | |
| Cmpd D 20 mg/kg | 9.3 +/− 0.5 | 15.4 +/− 0.6 | 45.6 +/− 1.8 |
| (n = 8) | | | |
| Cmpd C 20 mg/kg | 9.8 +/− 1.1 | 16.3 +/− 1.6 | 47.2 +/− 5.6 |
| (n = 8) | | | |

Values represent means +/− SD. Non-tumored vehicle was compared with tumored vehicle, and tumored treatment groups were compared with tumored vehicle.
#= pval < 0.05 vs. non-tumored vehicle, Student-Newman-Keuls (SNK) Method.
*= pval < 0.05 vs. tumored vehicle control.

As shown in Table 5, administration of compounds of the present invention to animals with cancer (i.e., a mouse xenograft model of cancer of anemia) resulted in increased RBC, HGB, and HCT levels compared to that observed in non-treated vehicle control animals with cancer. These results indicated that methods and compounds of the present invention are useful for effectively treating and preventing the development of anemia of cancer or cancer-related anemia.

### Example 4

### Compounds and Methods of the Invention Are Effective at Treating Anemia of Cancer

The effectiveness of methods and compounds of the present invention at treating or preventing the development of anemia of cancer was examined as follows. In this study, experiments were performed essentially as described above in Example 1, with the following modifications. Tumors used in this study were MDA-MB-435-GFP breast tumors, and surgical orthotopic tumor fragment implantation was done directly into breast tissue of female CD-1 nude animals. The number of animals in each group was 15.

In the MDA-MB-435-GFP breast orthotopic study, administration of Compound A (20 mg/kg or 60 mg/kg) was initiated 21 days after tumor fragment implantation to the mammary fat pad of female CD-1 nude mice; at this time, the volume of primary tumors had reached ˜150 mm³. After mean tumor volume in the vehicle control group exceeded 1 cm³, animals were sacrificed (33 days treatment) and hematocrit was measured.

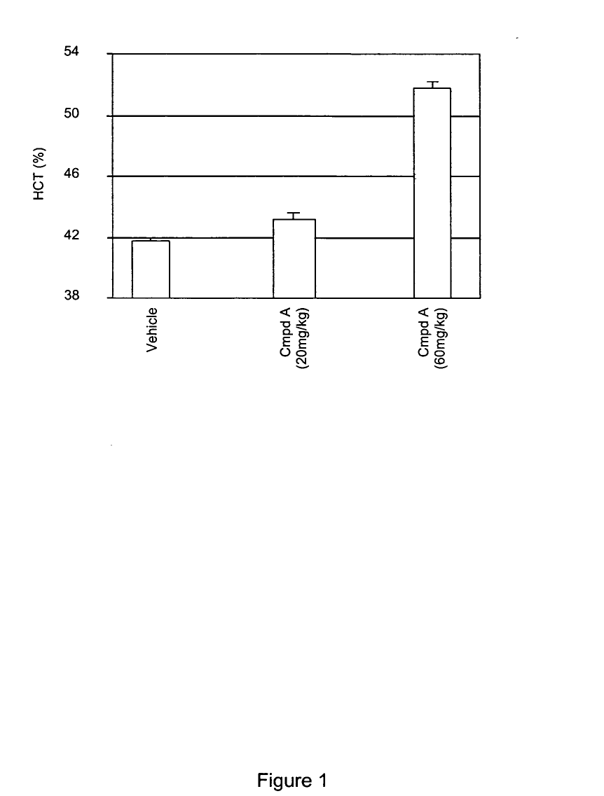

As shown in FIG. 1, hematocrit was reduced (<42%, see vehicle, FIG. 1) in vehicle-treated animals with tumors compared to the hematocrit observed in control (non-tumor) animals (hematocrit of non-tumor control animals was approximately 48%, data not shown). This result indicated that animals with cancer had decreased hematocrit levels compared to that in animals without cancer, indicating the animals had anemia of cancer.

Animals with cancer administered Compound A (20 mg/kg or 60 mg/kg) showed increased hematocrit levels compared to non-treated control animals with cancer. (See FIG. 1.) These results demonstrated that compounds and methods of the present invention are effective at treating anemia of cancer.

### Example 5

### Compounds and Methods of the Invention Are Effective at Treating Anemia of Cancer

The experiment was conducted as described above in Example 1 with the following modifications. Tumors used in this study were OVCAR3 ovarian tumors. OVCAR3 tumor fragments (1 mm³) were implanted subcutaneously into the flanks of female CB.17 SCID mice. The number of animals in each treatment group was 10. Treatment was initiated when tumors reached an approximate average size of 100 mm³. After 29 days treatment, animals were sacrificed and blood was collected for determination of hematocrit (HCT).

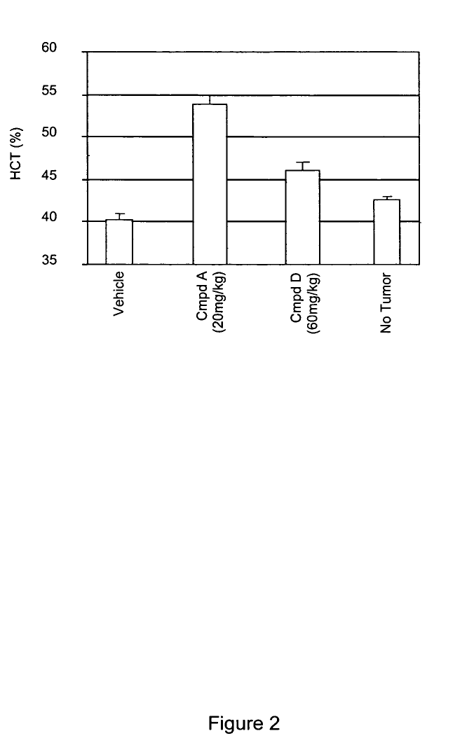

As shown in FIG. 2, the hematocrit of OVCAR3 tumor-bearing animals was reduced compared to that of non-tumor bearing animals. This indicated that OVCAR3 tumor-bearing animals had anemia of cancer. Administration of Compound A (60 mg/kg) or Compound D(60 mg/kg) increased hematocrit levels in OVCAR3 tumor-bearing animals. These results demonstrated that compounds and methods of the present invention are effective at treating anemia of cancer or cancer-related anemia.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method for treating or preventing anemia of cancer in a subject with cancer, the method comprising administering to the subject an effective amount of an agent that inhibits hypoxia inducible factor (HIF) hydroxylase activity, wherein the subject is refractory or is at risk for being refractory to recombinant human EPO therapy.

2. A method for increasing reticulocytes in a subject having anemia of cancer, the method comprising administering to the subject an effective amount of an agent that inhibits hypoxia inducible factor (HIF) hydroxylase activity, wherein the subject is refractory or is at risk for being refractory to recombinant human EPO therapy.

3. A method for increasing hemoglobin in a subject having anemia of cancer, the method comprising administering to the subject an effective amount of an agent that inhibits hypoxia inducible factor (HIF) hydroxylase activity, wherein the subject is refractory or is at risk for being refractory to recombinant human EPO therapy.

4. A method for increasing hematocrit in a subject having anemia of cancer, the method comprising administering to the subject an effective amount of an agent that inhibits hypoxia inducible factor (HIF) hydroxylase activity, wherein the subject is refractory or is at risk for being refractory to recombinant human EPO therapy.

5. A method for increasing red blood cell count in a subject having anemia of cancer, the method comprising administering to the subject an effective amount of an agent that inhibits hypoxia inducible factor (HIF) hydroxylase activity, wherein the subject is refractory or is at risk for being refractory to recombinant human EPO therapy.

6. The method of claim 1, wherein the agent is a 2-oxoglutarate mimetic.

7. The method of claim 1, wherein the agent is administered orally, systemically, intravenously, or by injection.
